(19) United States
(12) Patent Application Publication
(10) Pub. No.: US 2026/0076914 A1
(43) Pub. Date: Mar. 19, 2026

(54) IONIZABLE LIPIDS AND COMPOSITIONS COMPRISING SAME

(71) Applicant: BARCODE NANOTECH LTD., Nazareth (IL)

(72) Inventors: Ronen EAVRI, Binyamina (IL); Annie SABBAH, Haifa (IL); Felix BADINTER, Karmiel (IL); Aaron ROSENBLOOM, Karmiel (IL); Or ROZEN, Afula (IL); Eli EYDLIN, Nesher (IL)

(21) Appl. No.: 19/110,584

(22) PCT Filed: Sep. 11, 2023
(86) PCT No.: PCT/IL2023/050978

§ 371 Date: Mar. 11, 2025

Related U.S. Application Data
(60) Provisional application No. 63/405,459, filed on Sep. 11, 2022.

(51) Int. Cl. A61K 9/50; C07C 219/06; C07C 233/36; C07D 295/13; C07D 317/58

(52) U.S. Cl. CPC A61K 9/5015; C07C 219/06; C07C 233/36; C07D 295/13; C07D 317/58

(57) ABSTRACT

One or more ionizable lipid(s) and lipid nanoparticles comprising same are provided. Pharmaceutical compositions comprising the lipid nanoparticles encapsulating an active agent are also provided.

### CROSS-REFERENCE TO RELATED APPLICATIONS

This application claims the benefit of priority of U.S. Provisional Patent Application No. 63/405,459, titled “IONIZABLE LIPIDS AND COMPOSITIONS COMPRISING SAME”, filed Sep. 11, 2022, the contents of which are incorporated herein by reference in their entirety.

### FIELD OF INVENTION

The present invention is directed to ionizable lipids and lipid nanoparticles comprising same and use thereof in diagnostic, therapeutic and theranostic compositions.

### BACKGROUND OF THE INVENTION

New delivery methods for therapeutic and diagnostic compounds are in constant development. Although lipid-based nanoparticles are a well-known delivery modality, these agents are also constantly undergoing improvement. Among other concerns, the ability of a therapeutic carrier to effectively load the active agent, as well as any potential accessory agents such as nucleic acid molecules, is important for accurate dosing, decreasing drug loss/cost and streamlining methods of drug production.

In particular, lipids that are positively charged at low pH are highly useful for loading nucleic acids which have a net negative charge. However, positively charged lipids have been found to be toxic when administered systemically and also show poor biodistribution as they tend to adhere to cells and tissues. One solution to this problem is ionizable lipids, which are lipids that are charged at one pH and neutral at another. In particular, ionizable lipids that are positive at low pH (allowing for efficient loading) and neutral at physiological pH (lessening toxicity and improving biodistribution) are highly sought.

Although several such ionizable lipids are known, there is a constant need for new and superior ionizable lipids. The provision of new and superior ionizable lipids will greatly enhance the drug delivery arena and provide new compositions and modalities for delivery of active agents, therapeutics and diagnostic agents to subjects in general and specific locations in the body in particular.

### SUMMARY OF THE INVENTION

The present invention provides new compounds comprising an ionizable moiety. In particular, new ionizable lipids and nanoparticles comprising same are provided. Compositions comprising the nanoparticles, which are useful for therapeutic, diagnostic and theranostic methods are also provided.

According to a first aspect, there is provided a compound represented by Formula 1:

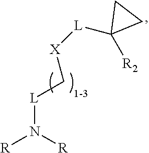

wherein each R independently comprises any of:

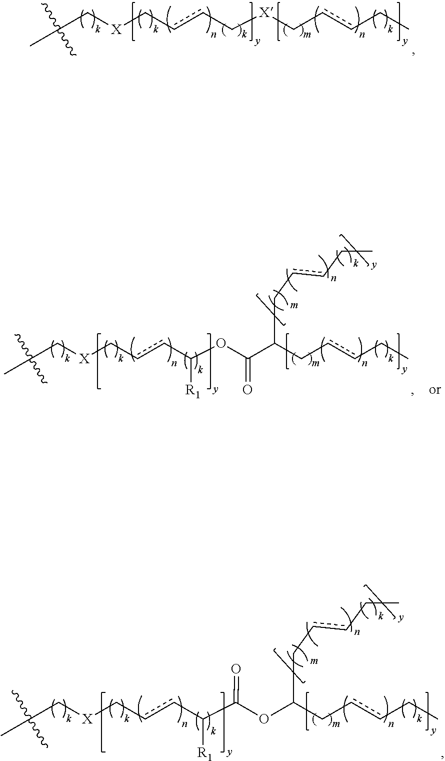

wherein k, and y, are integers each independently being between 1 and 10; n and m are integers each independently being between 0 and 10; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each X independently comprises CH₂, CHR₂, CHR₁, C(R₂)₂, C(R₁)₂, NR₁, NR₂, NH, O, S or is absent; each L independently is absent, or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C₁-C₃₀ alkyl, (ii) a C₁-C₃₀ alkenyl, (ii) a C₁-C₃₀ alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, or a combination thereof.

In one embodiment, the compound is or comprises any one of compounds of FIG. 1.

In another aspect, there is provided a compound represented by Formula 2:

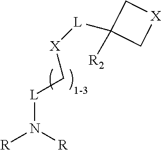

wherein each R independently comprises any of:

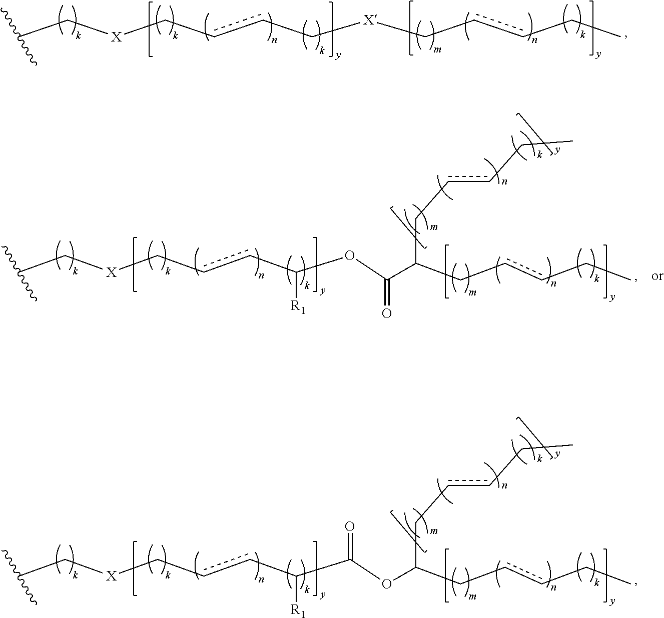

wherein k, and y, are integers each independently being between 1 and 10; n and m are integers each independently being between 0 and 10; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each X independently comprises CH₂, CHR₂, CHR₁, C(R₂)₂, C(R₁)₂, NR₁, NR₂, NH, O, S, or is absent; each L independently is absent, or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C₁-C₃₀ alkyl, (ii) a C₁-C₃₀ alkenyl, (ii) a C₁-C₃₀ alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof, wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In one embodiment, the compound is or comprises any one of compounds of FIG. 2.

In another aspect, there is provided a compound represented by Formula 3:

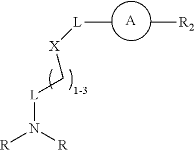

wherein each R independently comprises any of:

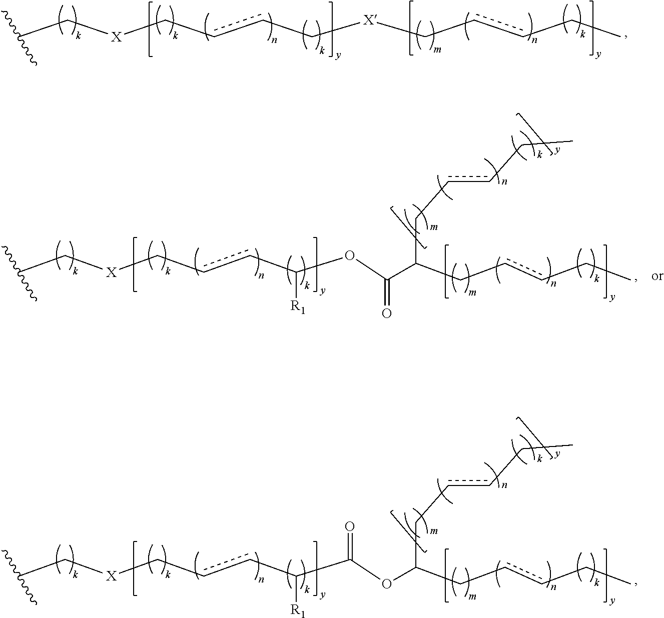

wherein k, and y, are integers each independently being between 1 and 10; n and m are integers each independently being between 0 and 10; A represents a 5-membered heteroaryl, 5-membered cycloalkyl optionally comprising an unsaturated bond, or a 5-membered heterocyclyl optionally comprising an unsaturated bond, each X independently comprises CH₂, CHR₂, C(R₂)₂, C(R₁)₂, CHR₁, NR₁, NR₂, NH, O, S, or is absent; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each L independently is absent, or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C₁-C₃₀ alkyl, (ii) a C₁-C₃₀ alkenyl, (ii) a C₁-C₃₀ alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof, wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In one embodiment, the compound is or comprises any one of compounds of FIG. 3.

In another aspect, there is provided a compound represented by Formula 4:

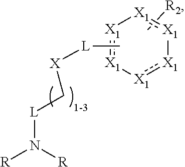

wherein each R independently comprises any of:

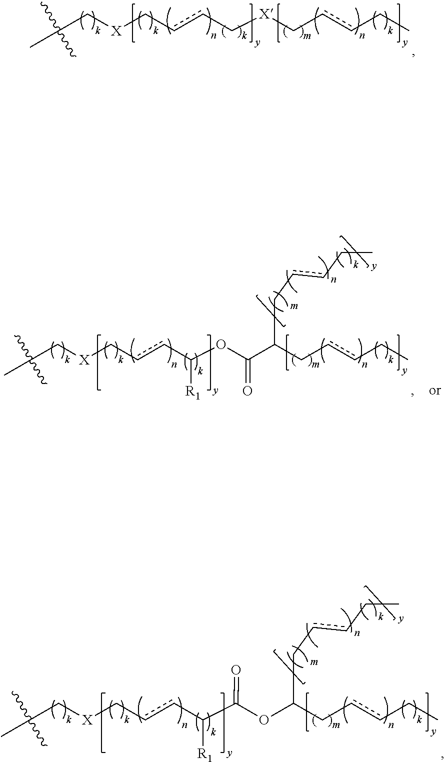

wherein k, and y, are integers each independently being between 1 and 10; n and m are integers each independently being between 0 and 10; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each X independently comprises CH₂, CHR₂, CHR₁, C(R₂)₂, C(R₁)₂, NR₁, NR₂, NH, O, S, or is absent; each X₁ independently comprises CH, CH₂, CHR₂, CHR₁, NR₁, NR₂, NH, N, O, or S; each L independently is absent or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C₁-C₃₀ alkyl, (ii) a C₁-C₃₀ alkenyl, (ii) a C₁-C₃₀ alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof, wherein each R′ independently represents hydrogen, or is selected from the group comprising optionally substituted C₁-C₁₀ alkyl, optionally substituted C1-C30 alkyl, optionally substituted C1-C30 alkenyl, optionally substituted C1-C30 alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In one embodiment, between 1 and 3 of the X₁ is independently selected from NR₁, NR₂, NH, N, O, or S, and the additional X₁ is independently selected from CH, CH₂, CHR₂, or CHR₁.

In one embodiment, the compound is or comprises any one of compounds of FIG. 4.

In another aspect, there is provided a compound represented by Formula 5:

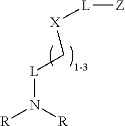

wherein Z represents (i) an optionally substituted bicyclic aliphatic ring optionally comprising between 1 and 4 X₁, or (ii) an optionally substituted bicyclic ring optionally comprising between 1 and 4 X₁, wherein at least one ring is aromatic, each R independently comprises any of:

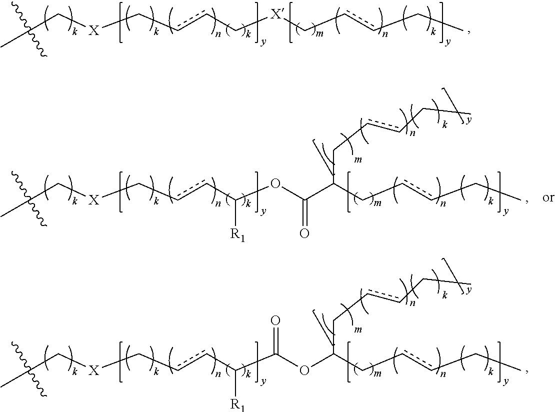

wherein k, and y, are integers each independently being between 1 and 10; n and m are integers each independently being between 0 and 10; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each X independently comprises CH₂, CHR₂, CHR₁, C(R₂)₂, C(R₁)₂, NR₁, NR₂, NH, O, S, or is absent; each X₁ independently comprises CH, CH₂, CHR₂, CHR₁, NR₁, NR₂, NH, N, O, or S; each L independently is absent or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C₁-C₃₀ alkyl, (ii) a C₁-C₃₀ alkenyl, (ii) a C₁-C₃₀ alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof, wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In one embodiment, Z comprises a 7-12 membered bicyclic ring optionally substituted with one or more R₂.

In one embodiment, the 7-12 bicyclic ring optionally comprises between 1 and 4 X₁, including any range between.

In one embodiment, the compound is or comprises any one of compounds of FIG. 5.

In another aspect, there is provided a compound represented by Formula 6:

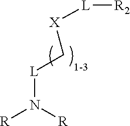

wherein each R independently comprises any of:

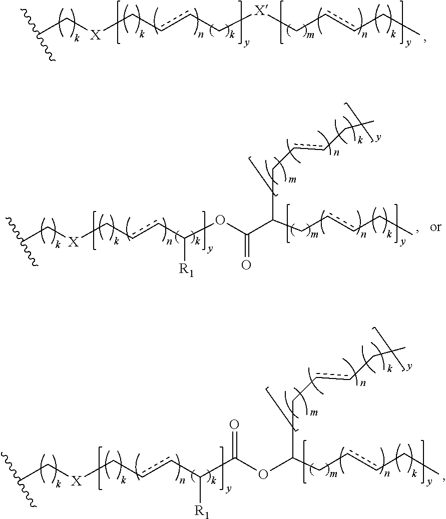

wherein k, and y, are integers each independently being between 1 and 10; n and m are integers each independently being between 0 and 10; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each X independently comprises CH₂, CHR₂, C(R₂)₂, CHR₁, C(R₁)₂, NR₁, NR₂, NH, O, S, or is absent; each L independently is absent or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; each R₁ is H or independently comprises (i) a C₁-C₃₀ alkyl, (ii) a C₁-C₃₀ alkenyl, (ii) a C₁-C₃₀ alkynyl, each of (i), (ii) and (iii) optionally comprising one or more heteroatoms; wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof, and wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In one embodiment, the compound is or comprises any one of compounds of FIG. 6.

In one embodiment, each R is independently

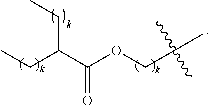

In one embodiment, each k is between 4 and 6, including any range between.

In another aspect, there is provided a nanoparticle comprising a core and a shell; the shell comprises a lipid, and at least one compound of the invention; the core comprises an active agent; and wherein an average size of the nanoparticle is in a range between 10 and 1000 nm.

In another aspect, there is provided a pharmaceutical composition that comprises a plurality of the nanoparticles of any one of the invention and a pharmaceutically acceptable carrier.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 represents the molecular structure of the exemplary compounds according to Formula 1. “?” indicates a chiral atom.

FIG. 2 represents the molecular structure of the exemplary compounds according to Formula 2. “?” indicates a chiral center.

FIG. 3 represents the molecular structure of the exemplary compounds according to Formula 3. “?” indicates a chiral center.

FIG. 4 represents the molecular structure of the exemplary compounds according to Formula 4. “?” indicates a chiral center.

FIG. 5 represents the molecular structure of the exemplary compounds according to Formula 5. “?” indicates a chiral center.

FIG. 6 represents the molecular structure of the exemplary compounds according to Formula 6. “?” indicates a chiral center.

FIG. 7 is a scheme representing exemplary strategies (Route 1 or Route 2) for synthesizing the compounds represented in FIGS. 1-6.

FIGS. 8A-8C are bar graphs demonstrating in-vivo accumulation of lipid nanoparticles (LNP) of the invention (disclosed in Example 2) normalized to control. Formulation 40-2 (8A), Formulation 40-6 (8B), Formulation 40-7 (8C).

### DETAILED DESCRIPTION OF THE INVENTION

According to a first aspect, there is provided a compound comprising an ionizable moiety (e.g., head group) covalently bound to a lipophilic tail (e.g., hydrocarbon based chain), wherein the ionizable moiety is as presented in any one of Formulae 1-6 disclosed herein. In some embodiments, the compound is an amphiphilic compound. In some embodiments, the compound (optionally together with additional liposome forming lipid(s)) is capable of spontaneously self-assembling to form a nanoparticle (e.g., a liposome) in an aqueous solution.

As used herein, the term “liposome forming lipid” encompasses lipids (e.g., phospholipids) which upon dispersion or dissolution thereof in an aqueous solution at a temperature above a transition temperature (Tₘ), undergo self-assembly so as to form stable vesicles (e.g., lipid nanoparticles). As used herein, the term Tm refers to a temperature at which the lipids undergo phase transition from solid (ordered phase, also termed as a gel phase) to a fluid (disordered phase, also termed as fluid crystalline phase). Tm also refers to a temperature (or to a temperature range) at which the maximal change in heat capacity occurs during the phase transition.

In some embodiments, the ionizable moiety is capable of undergoing ionization (protonation, or positive ionization) within a solution having a pH value below the pKa value of the ionizable moiety. In some embodiments, the ionizable moiety is capable of undergoing protonation within a solution having a pH value below the pKa value of the ionizable moiety. In some embodiments, at least 50 mol % of the ionizable moieties are positively charged (or protonated) within a solution having a pH value below the pKa value of the ionizable moiety.

In some embodiments, the pKa value of the ionizable moiety is between 2 and 11, including any range between. In some embodiments, the pKa value of the ionizable moiety is between 2 and 11, between 3 and 11, between 3 and 9, between 3 and 8, between 3 and 7, between 6 and 11, between 8 and 11, between 8 and 10, between 9 and 11, including any range between.

In some embodiments, the compound of the invention is represented by Formula 1:

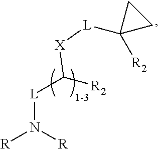

wherein each R independently comprises any of:

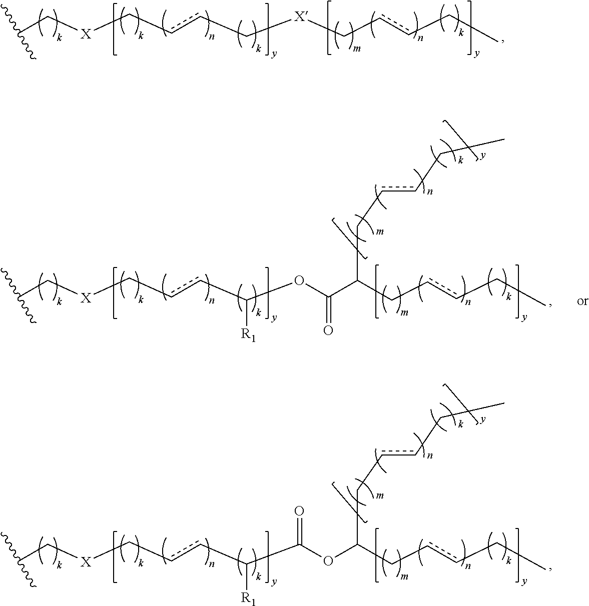

wherein k, and y, are integers each independently being between 1 and 10; n and m are integers each independently being between 0 and 10; each X independently comprises CH₂, C(R₂)₂, C(R₁)₂, CHR₂, CHR₁, NR₁, NR₂, NH, O, S or is absent; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each L independently is absent, or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C₁-C₃₀ alkyl, (ii) a C₁-C₃₀ alkenyl, (ii) a C₁-C₃₀ alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In some embodiments, the compound of the invention is represented by Formula 1, wherein each R is independently

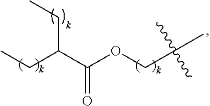

wherein each k is independently as described herein. In some embodiments, each k is between 3 and 10, between 4 and 8, between 4 and 6, including any range between.

In some embodiments, the compound of the invention is represented by Formula 1A:

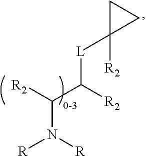

wherein each R is independently

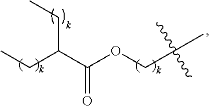

wherein each k is independently as described herein; and wherein L and R2 are as described herein.

In some embodiments, the compound of the invention is represented by Formula 1B:

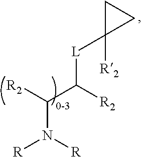

wherein each R is independently

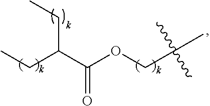

wherein each k is independently as described herein; wherein R′₂ is selected from H, hydroxy and hydroxy(C₁-C₆ alkyl); wherein L is absent (i.e. a bond), or represents a functional group selected from an ester, a carbonyl, an amide, NR₁, O, and S, or any combination thereof as allowed by valency; and wherein R₂ is as described herein.

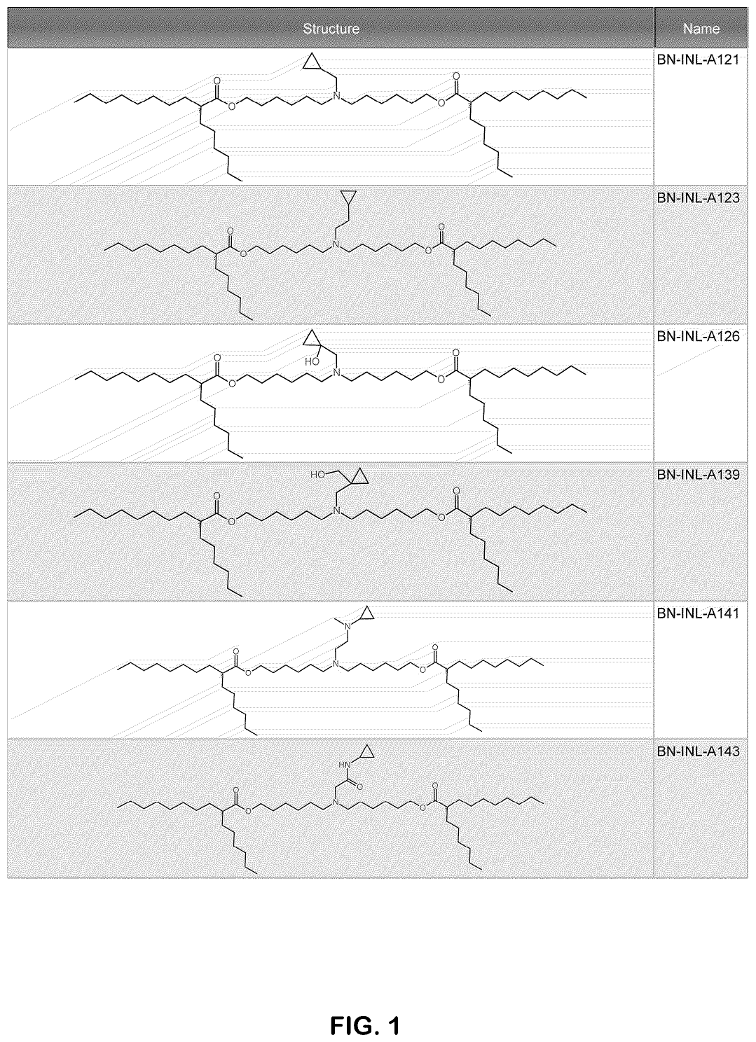

In some embodiments, the compound of the invention represented by Formula 1 is or comprises any of the compounds presented in FIG. 1.

In some embodiments, the compound of the invention is represented by Formula 2:

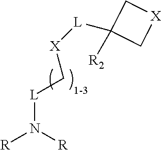

### or by Formula 2A:

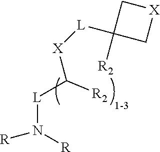

wherein each R independently comprises any of:

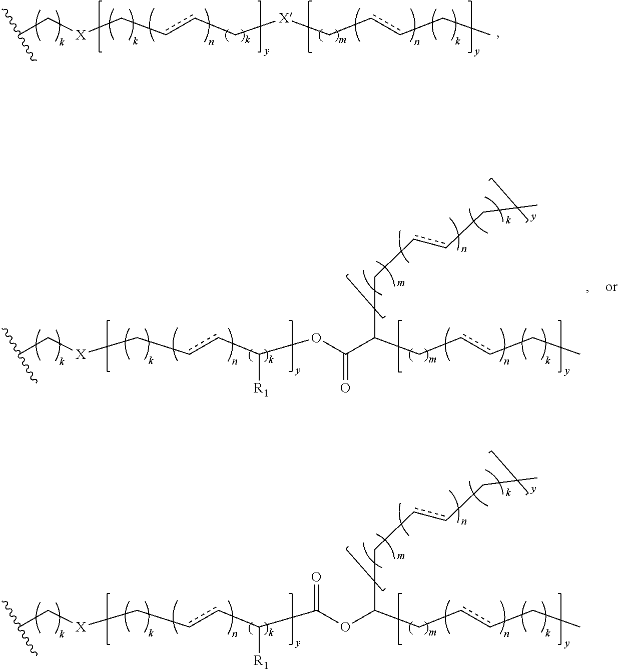

wherein k, and y, are integers each independently being between 1 and 10; n and m are integers each independently being between 0 and 10; each X independently comprises CH₂, CHR₂, CHR₁, C(R₂)₂, C(R₁)₂, NR₁, NR₂, NH, O, S, or is absent; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each L independently is absent, or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C₁-C₃₀ alkyl, (ii) a C₁-C₃₀ alkenyl, (ii) a C₁-C₃₀ alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof, wherein each R′ independently represents hydrogen, or is selected from the group comprising optionally substituted C₁-C₁₀ alkyl, optionally substituted C1-C30 alkyl, optionally substituted C1-C30 alkenyl, optionally substituted C1-C30 alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl —SR′, or a combination thereof.

In some embodiments, the compound of the invention is represented by Formula 2, wherein each R is independently

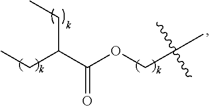

wherein each k is independently as described herein. In some embodiments, each k is between 3 and 10, between 4 and 8, between 4 and 6, including any range between.

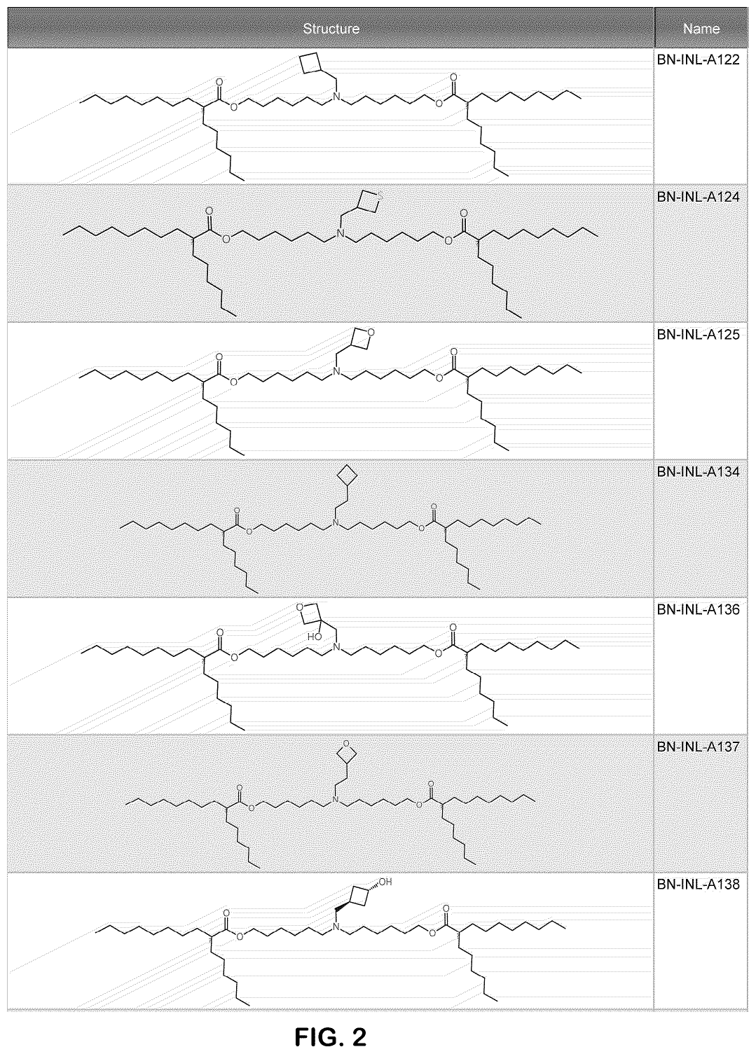

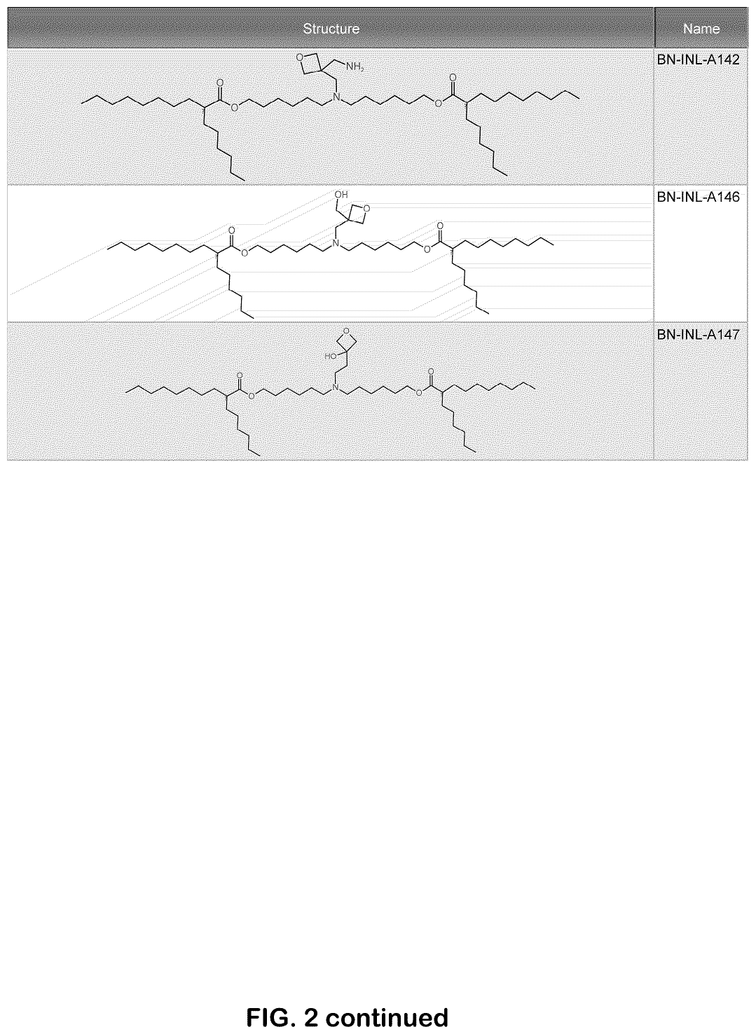

In some embodiments, the compound of the invention represented by Formula 2 is or comprises any of the compounds presented in FIG. 2.

In some embodiments, the compound of the invention is represented by Formula 3:

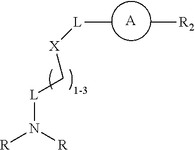

wherein each R independently comprises any of:

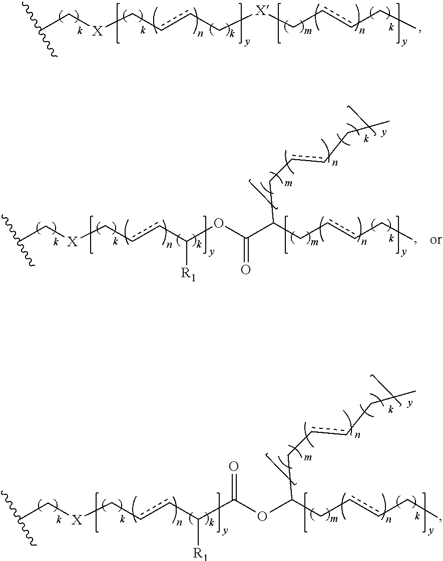

wherein k, and y, are integers each independently being between 1 and 10; n and m are integers each independently being between 0 and 10; A represents a 5-membered heteroaryl, 5-membered cycloalkyl optionally comprising an unsaturated bond, or a 5-membered heterocyclyl optionally comprising an unsaturated bond, each X independently comprises CH₂, CHR₂, CHR₁, C(R₂)₂, C(R₁)₂, NR₁, NR₂, NH, O, S, or is absent; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each L independently is absent, or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C1-C30 alkyl, (ii) a C1-C30 alkenyl, (ii) a C1-C30 alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NIINR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C1-C30 alkyl, optionally substituted C1-C30 alkenyl, optionally substituted C1-C30 alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In some embodiments, the compound of the invention is represented by Formula 3A:

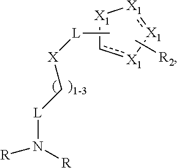

wherein each X₁ independently comprises CH, CH₂, CHR₂, CHR₁, NR₁, NR₂, NH, O, N, or S; and wherein R, L, X and R² are as disclosed hereinabove.

In some embodiments, the compound of the invention is represented by Formula 3 or 3A, wherein each R is independently

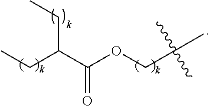

wherein each k is independently as described herein. In some embodiments, each k is between 3 and 10, between 4 and 8, between 4 and 6, including any range between.

In some embodiments, the compound of Formula 3 comprises a compound represented by Formula 3B:

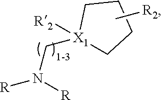

wherein X1 is C or N, as allowed by valency; wherein each R is independently

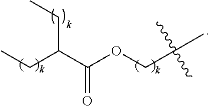

wherein each k is independently as described herein; wherein R′₂ is absent or comprises H, OR′, SR′, or N(R′)₂; and wherein R₂ is as disclosed hereinabove. In some embodiments, the compound of Formula 3 comprises a compound represented by Formula 3B, wherein X1 is N and wherein R′₂ is absent. In some embodiments, the compound of Formula 3 comprises a compound represented by Formula 3B, wherein X1 is C and wherein R′₂ comprises H, OR′, SR′, or N(R′)₂.

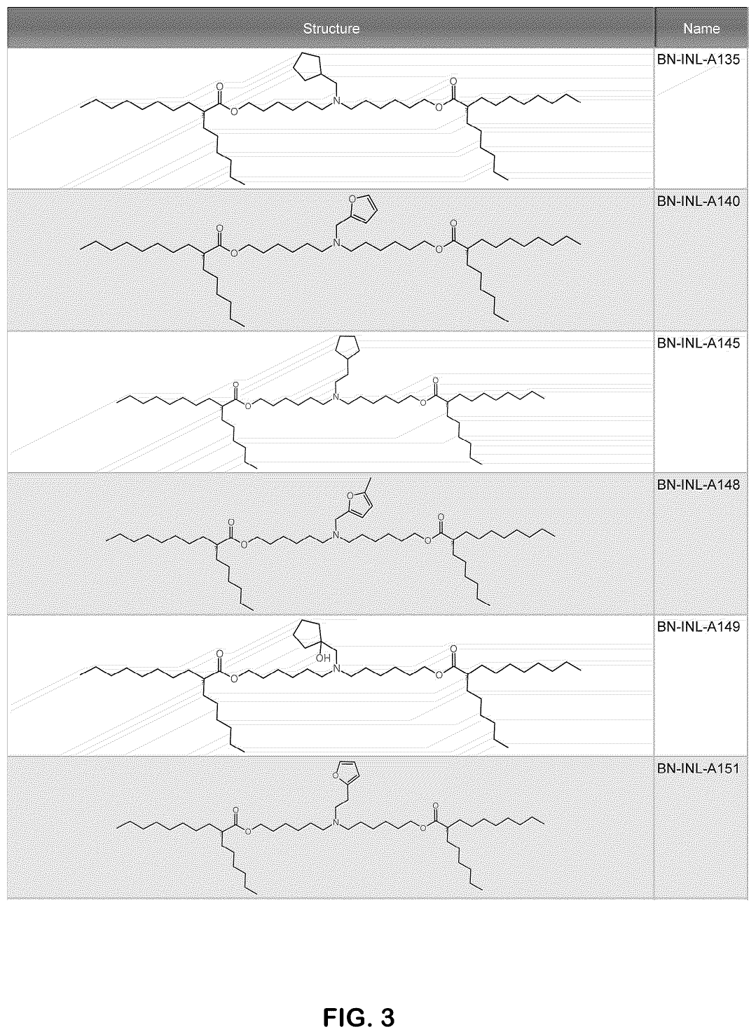

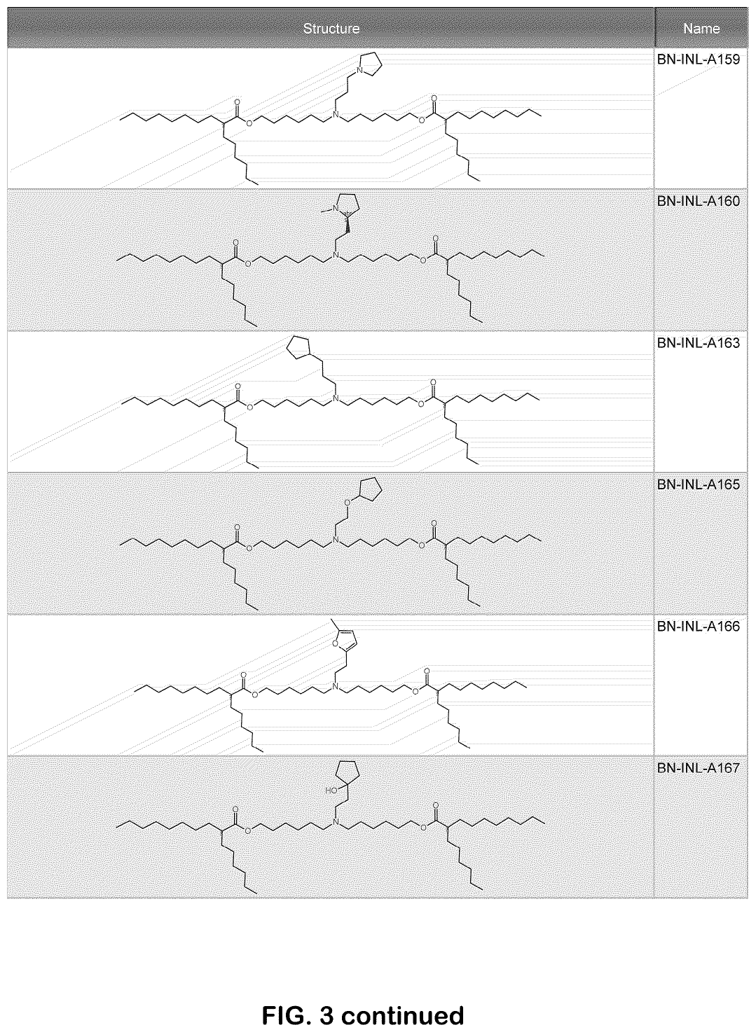

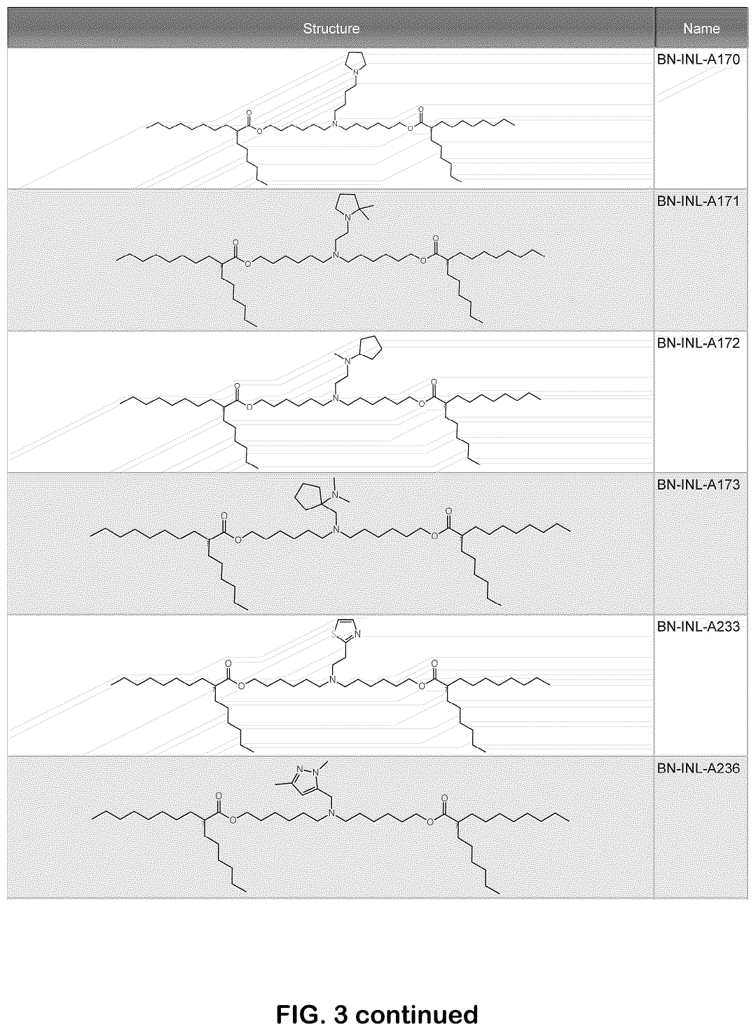

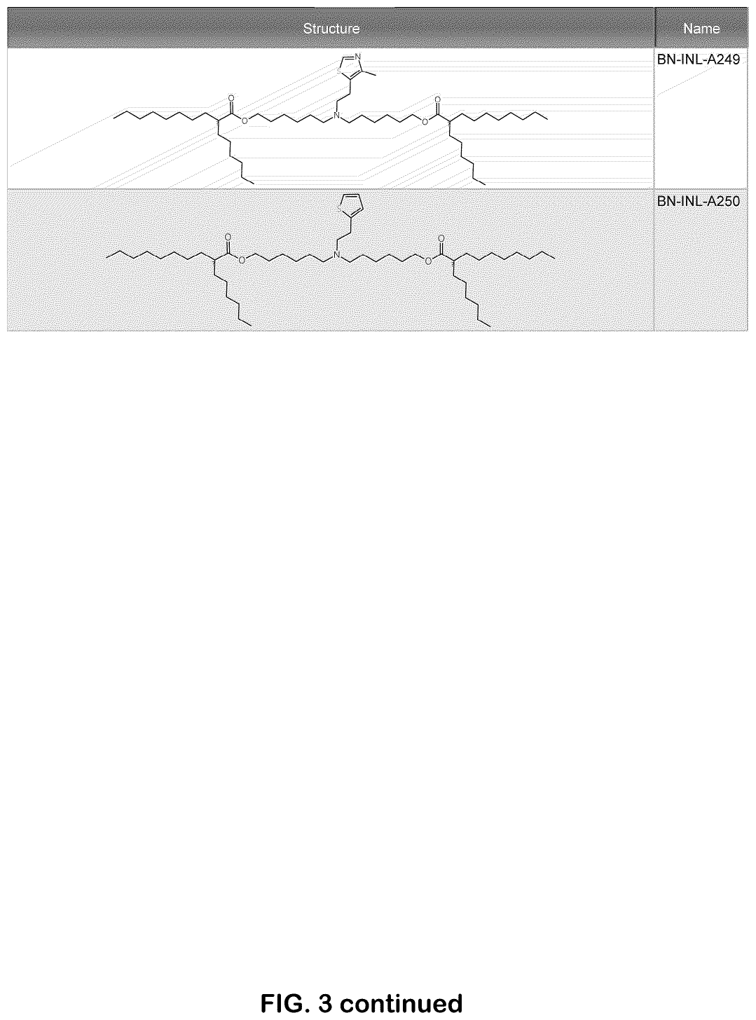

In some embodiments, the compound of the invention represented by Formulae 3-3B is or comprises any of the compounds presented in FIG. 3.

In some embodiments, the compound of the invention is represented by Formula 4:

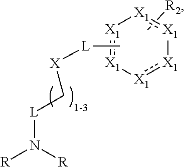

wherein each R independently comprises any of:

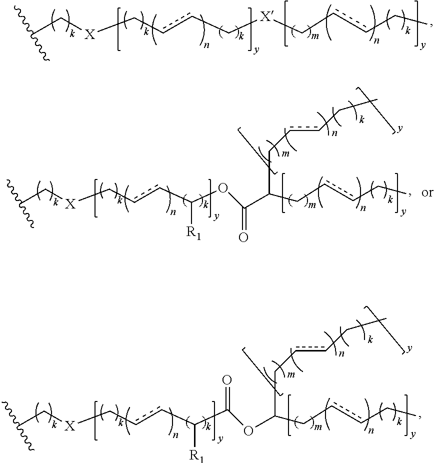

wherein k, and y, are integers each independently being between 1 and 10; n and m are integers each independently being between 0 and 10; each X independently comprises CH₂, CHR₂, C(R₂)₂, C(R₁)₂, CHR₁, NR₁, NR₂, NH, O, S, or is absent; each X₁ independently comprises CH, CH₂, CHR₂, CHR₁, NR₁, NR₂, NH, N, O, or S; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each L independently is absent or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C₁-C₃₀ alkyl, (ii) a C₁-C₃₀ alkenyl, (ii) a C₁-C₃₀ alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NIINR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In some embodiments, the compound of the invention is represented by Formula 4, wherein 1, 2, or 3 of the X₁ is/are independently selected from NR₁, NR₂, NH, N, O, or S, and the additional X₁ is/are independently selected from CH, CH₂, CHR₂, or CHR₁.

In some embodiments, the compound of the invention is represented by Formula 4, wherein each R is independently

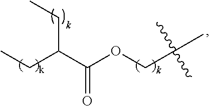

wherein each k is independently as described herein. In some embodiments, each k is between 3 and 10, between 4 and 8, between 4 and 6, including any range between.

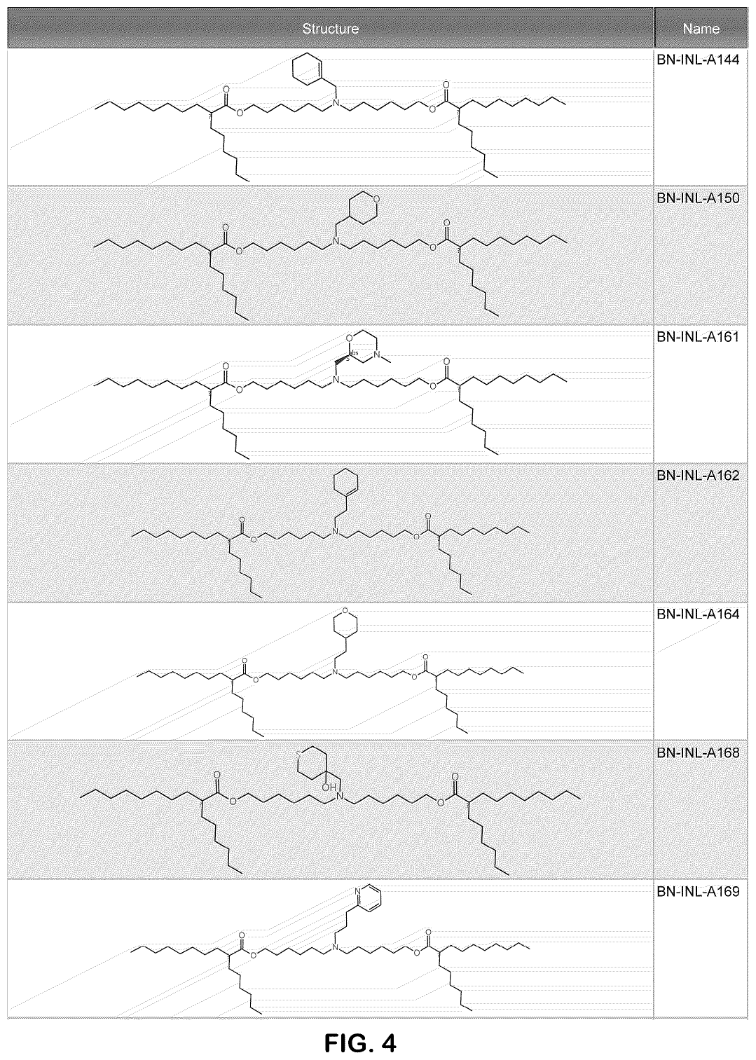

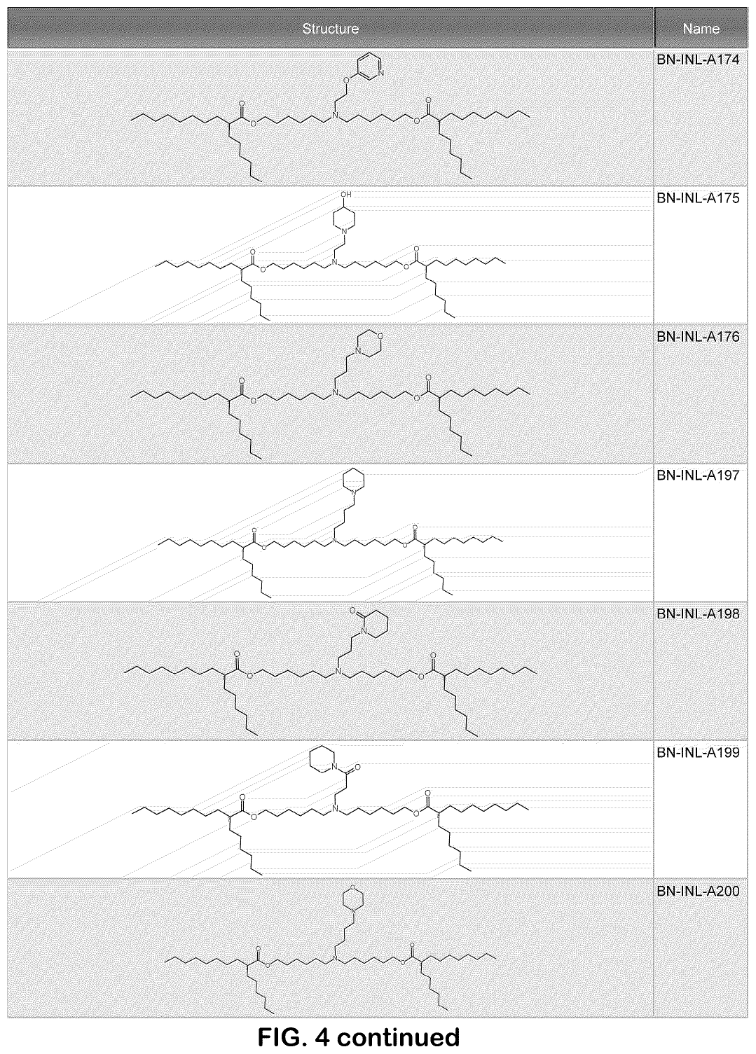

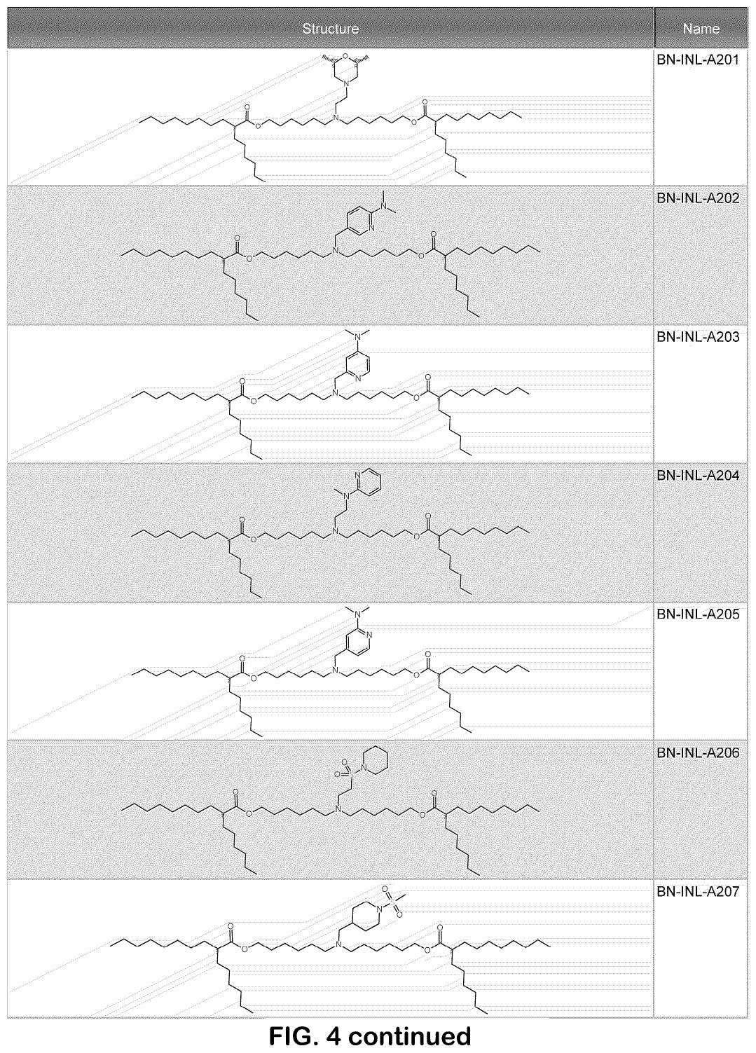

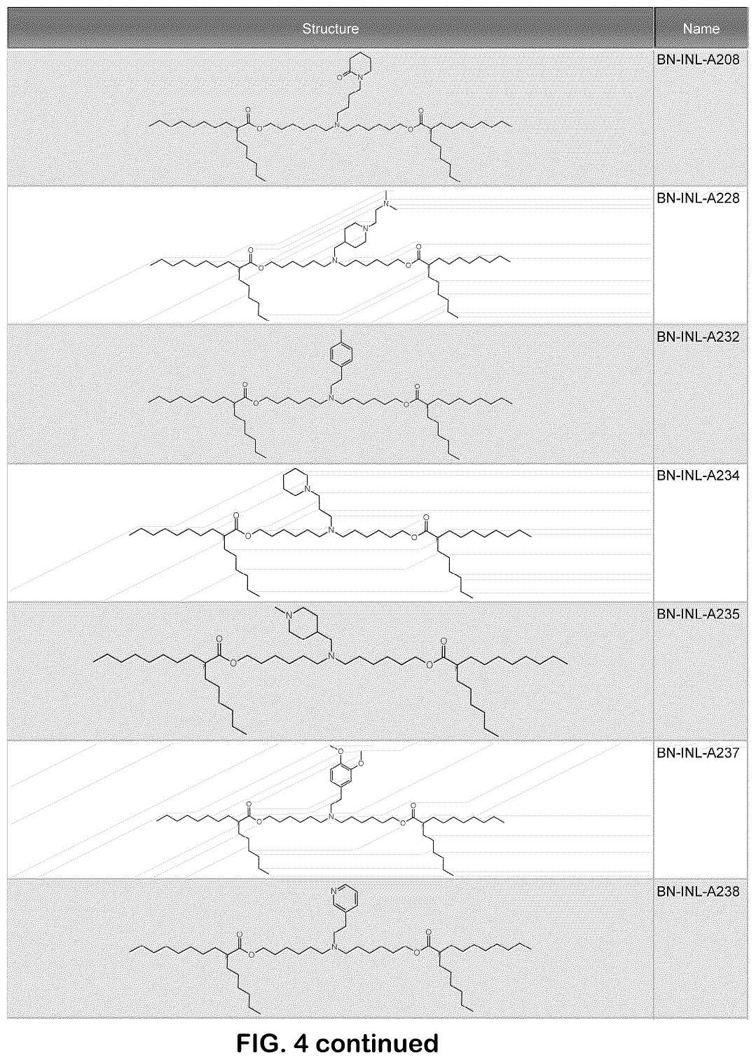

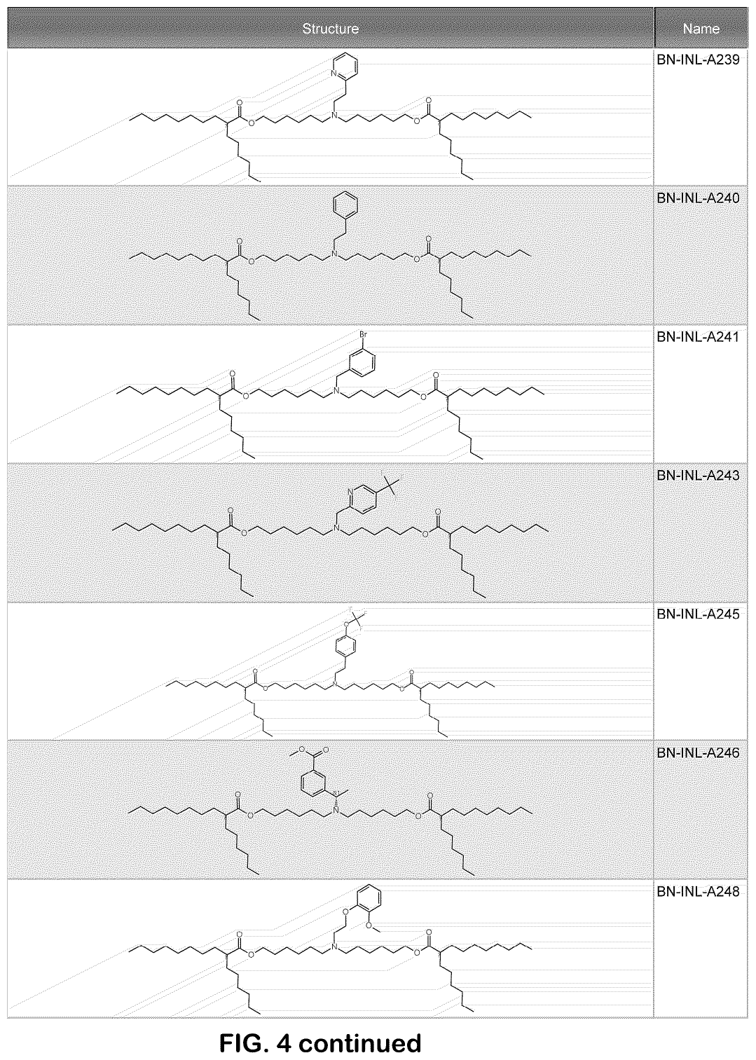

In some embodiments, the compound of the invention represented by Formula 4 is or comprises any of the compounds presented in FIG. 4.

In some embodiments, the compound of the invention is represented by Formula 5:

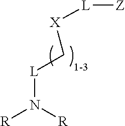

wherein Z represents (i) an optionally substituted bicyclic aliphatic ring optionally comprising between 1 and 4 X₁, (ii) an optionally substituted bicyclic ring optionally comprising between 1 and 4 X₁, wherein at least one ring is aromatic; (iii) a polycyclic aromatic, heteroaromatic, or aliphatic ring (e.g. adamantly); each R independently comprises any of:

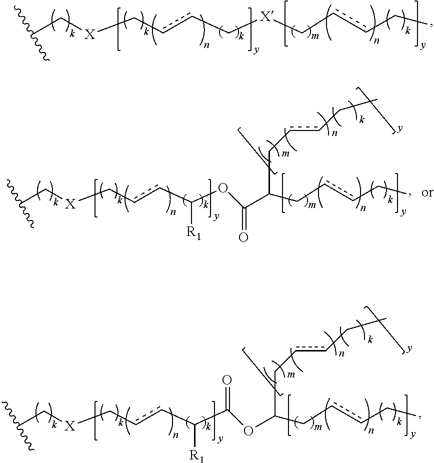

wherein k, and y, are integers each independently being between 1 and 10; n and m are integers each independently being between 0 and 10; each X independently comprises CH₂, CHR₂, C(R₂)₂, C(R₁)₂, CHR₁, NR₁, NR₂, NH, O, S, or is absent; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each X₁ independently comprises CH, CH₂, CHR₂, CHR₁, NR₁, NR₂, NH, N, O, or S; each L independently is absent or represents a (C1-C5) alkyl, an ester, a carbonyl, an amide, CHR₁, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C1-C30 alkyl, (ii) a C1-C30 alkenyl, (ii) a C1-C30 alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NIINR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In some embodiments, the term “bicyclic ring” encompasses a fused ring (fused aromatic, heteroaromatic ring and/or mixed fused aliphatic-aromatic ring), spirocyclic ring, a bridged ring, a dicyclyl (two aromatic rings such as a biaryl, one aromatic and one aliphatic ring, and/or aliphatic rings joined by a single carbon-carbon bond). In some embodiments, the term “substituted” encompasses substitution by one or more R₂, wherein R₂ is as described hereinabove.

In some embodiments, Z comprises 7-12 membered bicyclic ring optionally substituted with one or more R₂, wherein the 7-12 membered bicyclic ring optionally comprises between 1 and 4 X₁ or between 1 and 2 X₁ including any range between, wherein each X₁ independently is as described herein.

In some embodiments, Z comprises 7-12 membered polycyclic ring (e.g. tri-cyclic ring) optionally substituted with one or more R₂, wherein each ring of the 7-12 membered polycyclic ring optionally comprises between 1 and 4 X₁ or between 1 and 2 X₁ including any range between, wherein each X₁ independently is as described herein.

In some embodiments, Z comprises:

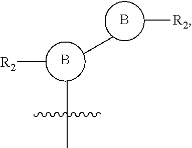

wherein each R² is as described herein; and wherein each B independently comprises any of: (i) an aliphatic ring optionally comprising between 1 and 4 X₁, (ii) an aromatic or heteroaromatic ring, (iii) a bicyclic aliphatic, bicyclic aromatic/heteroaromatic, or a bicyclic mixed aromatic/heteroaromatic-aliphatic ring, wherein each ring optionally comprising between 1 and 4 X₁; (iv) a polycyclic aromatic, heteroaromatic, or aliphatic ring (e.g. adamantyl); and wherein X1 represents N, NH, O or S.

Exemplary Z include but are not limited to indole, isoindole, benzofuran, adamantyl, benzothiophene, benzotriazole, quinoline, chromene, chroman, quinazoline, imidazopyridine, pyrazollopyridines, oxazolopyridines, isoxazolopyridines, thiazolopyridines, isothiazolopyridines, pyrimidine, purine. Other bicyclic rings (such as fused aromatic rings) are well-known in the art.

In some embodiments, the compound of the invention is represented by Formula 5, wherein each R is independently

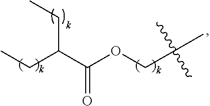

wherein each k is independently as described herein. In some embodiments, each k is between 3 and 10, between 4 and 8, between 4 and 6, including any range between.

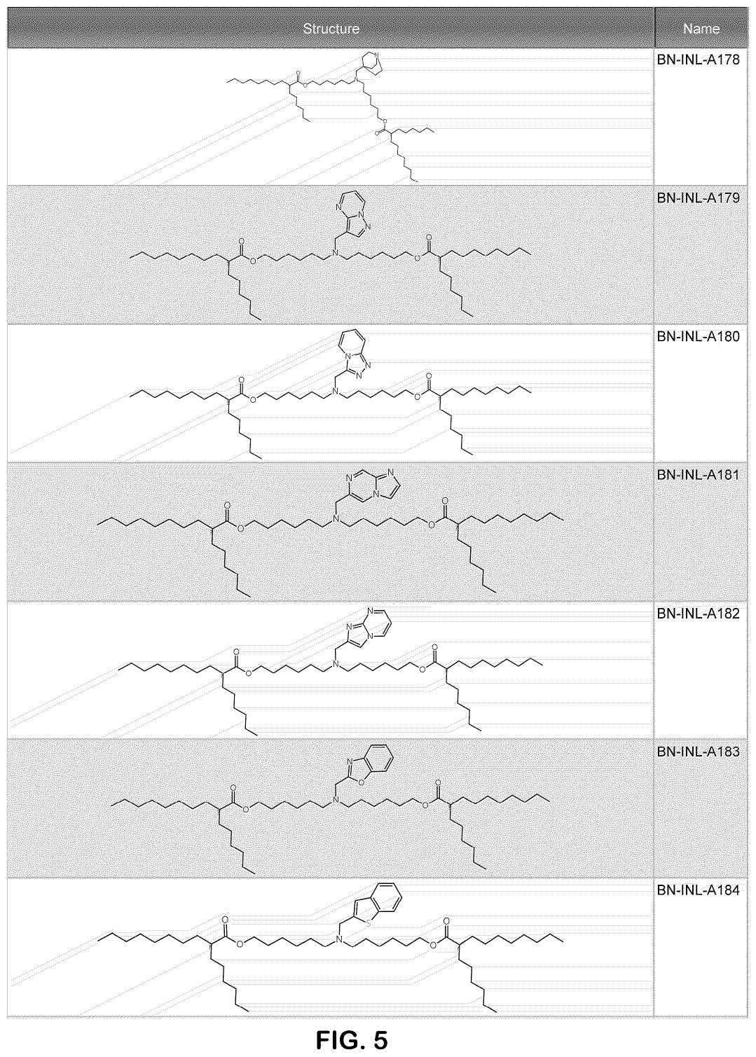

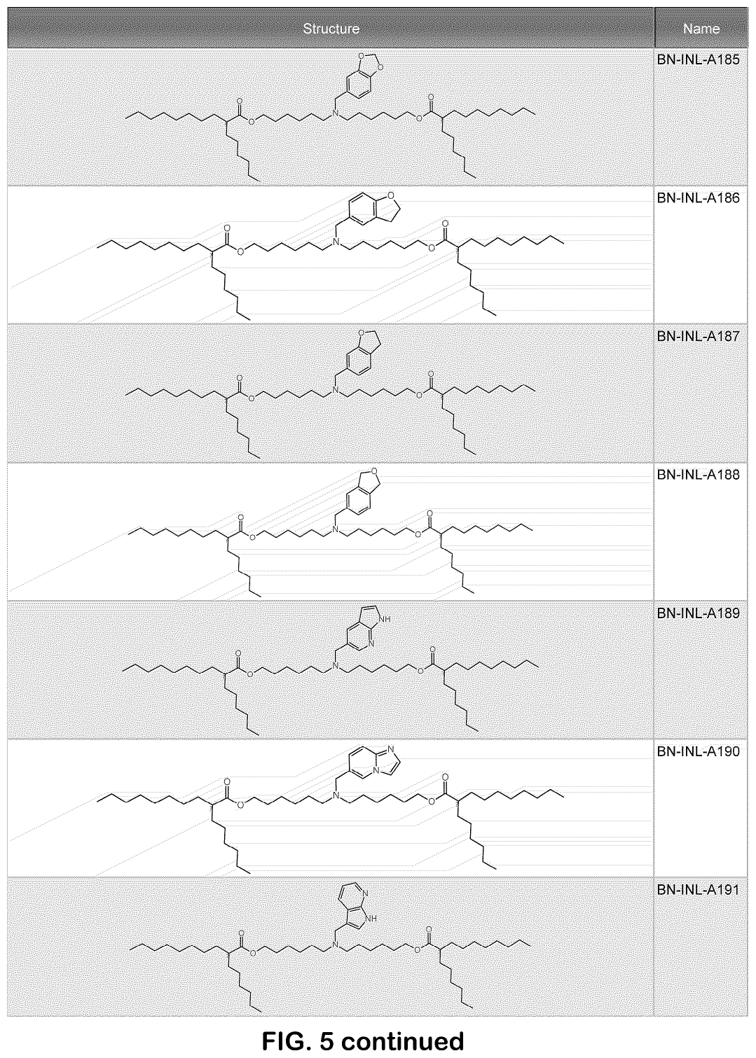

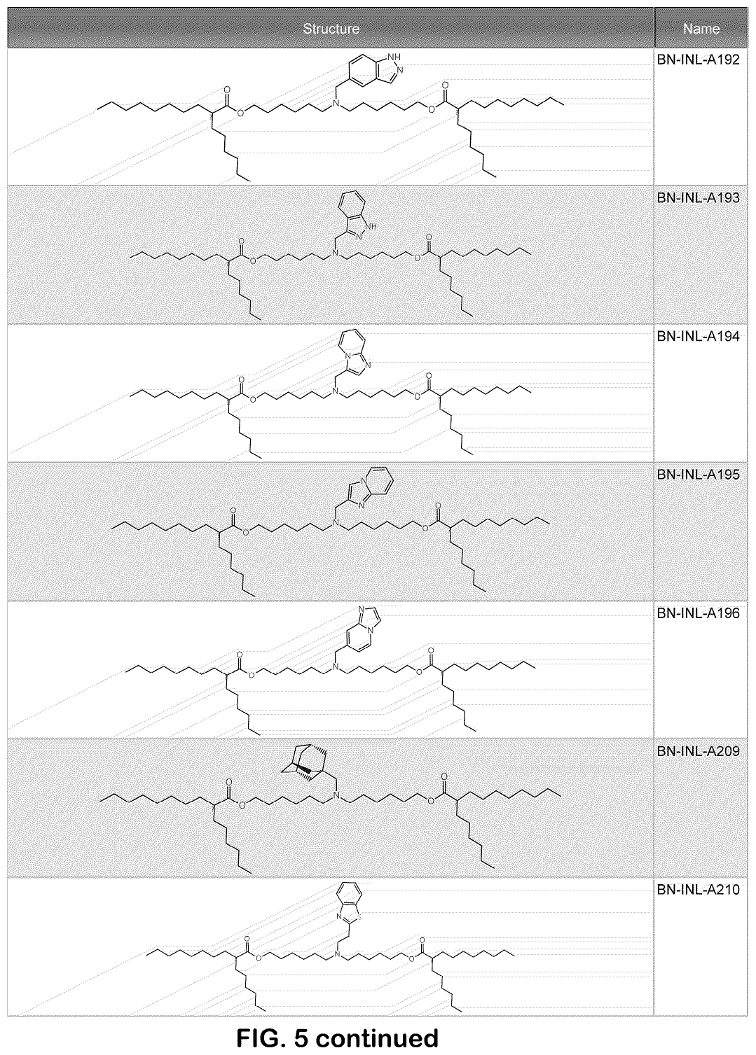

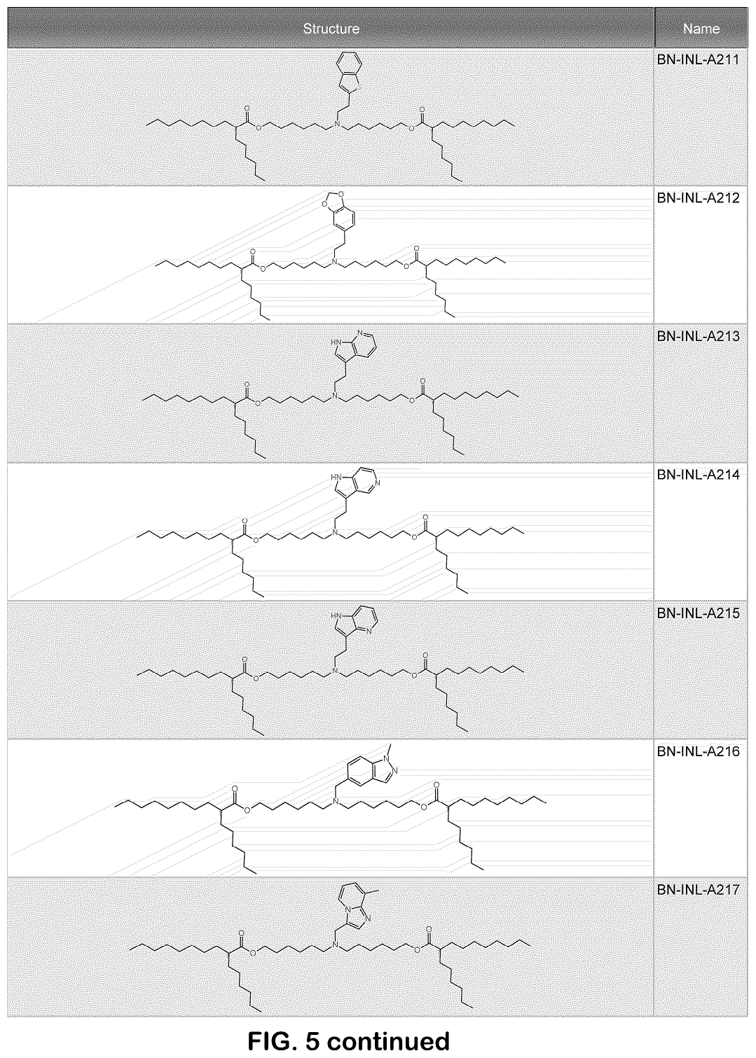

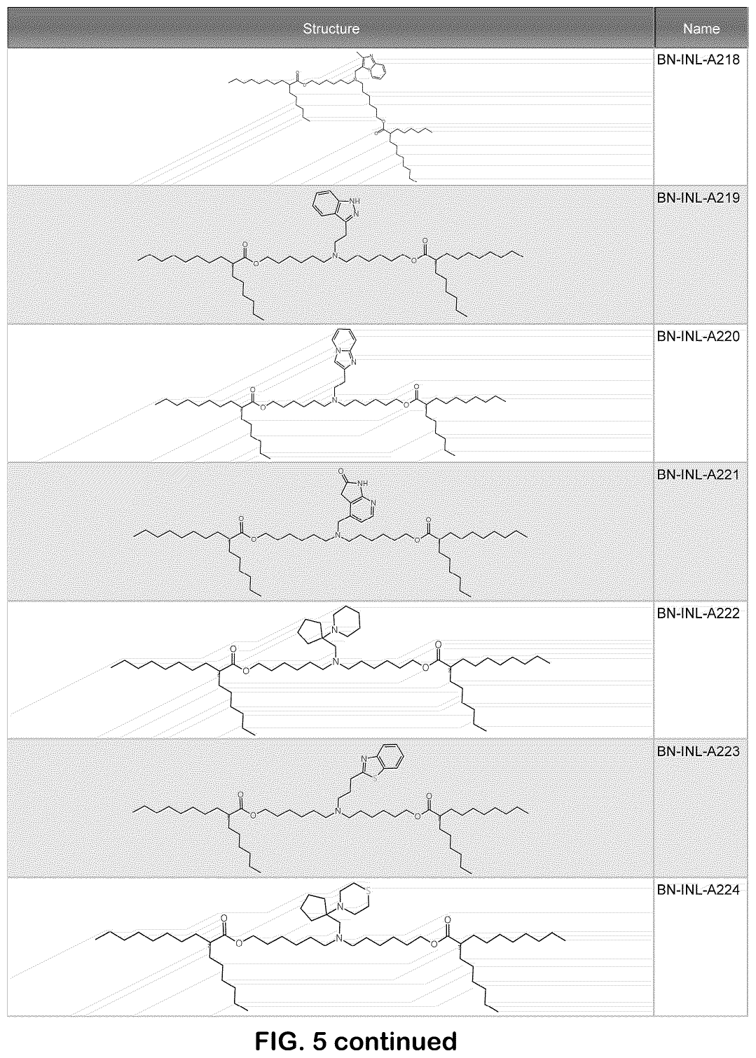

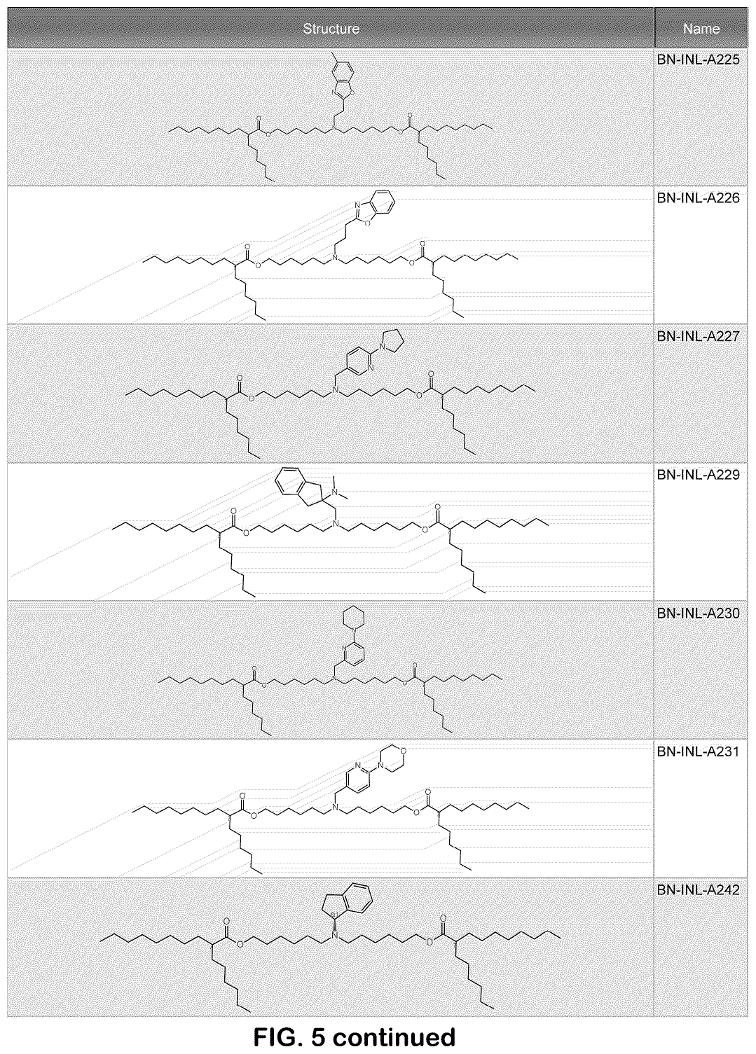

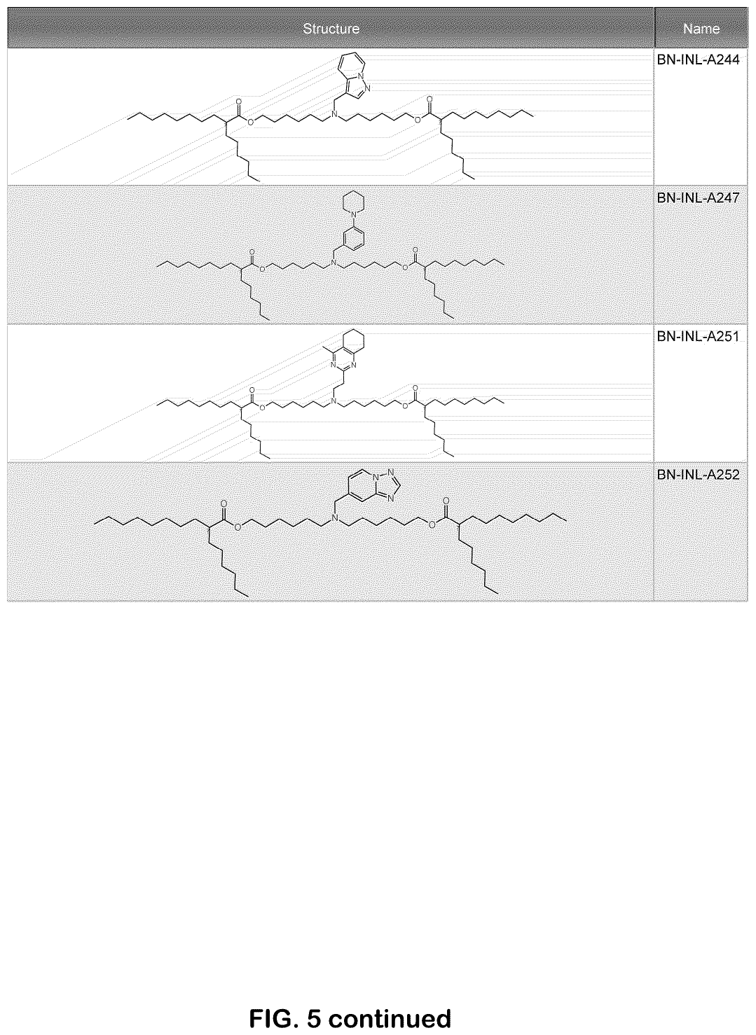

In some embodiments, the compound of the invention represented by Formula 5 is or comprises any of the compounds presented in FIG. 5.

In some embodiments, the compound of the invention is represented by Formula 6:

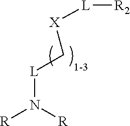

wherein each R independently comprises any of:

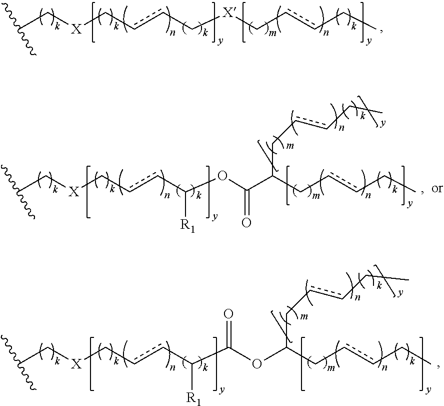

wherein k, and y, are integers each independently being between 1 and 10; n and m are integers each independently being between 0 and 10; each X independently comprises CH₂, CHR₂, CHR₁, C(R₂)₂, C(R¹)₂, NR₁, NR₂, NH, O, S, or is absent; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each L independently is absent or represents a (C1-C5) alkyl, an ester, a carbonyl, an amide, CHR₁, C(R₁)₂, NR₁, NH, O, S, —NH(C₁-C₆ alkyl), —N(C1-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C1-C30 alkyl, (ii) a C1-C30 alkenyl, (ii) a C1-C30 alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof, wherein each R′ independently represents hydrogen, or is selected from the group comprising optionally substituted C₁-C₁₀ alkyl, optionally substituted C1-C30 alkyl, optionally substituted C1-C30 alkenyl, optionally substituted C1-C30 alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof.

In some embodiments, the compound of the invention is represented by Formula 6, wherein each R is independently

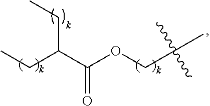

wherein each k is independently as described herein. In some embodiments, each k is between 3 and 10, between 4 and 8, between 4 and 6, including any range between.

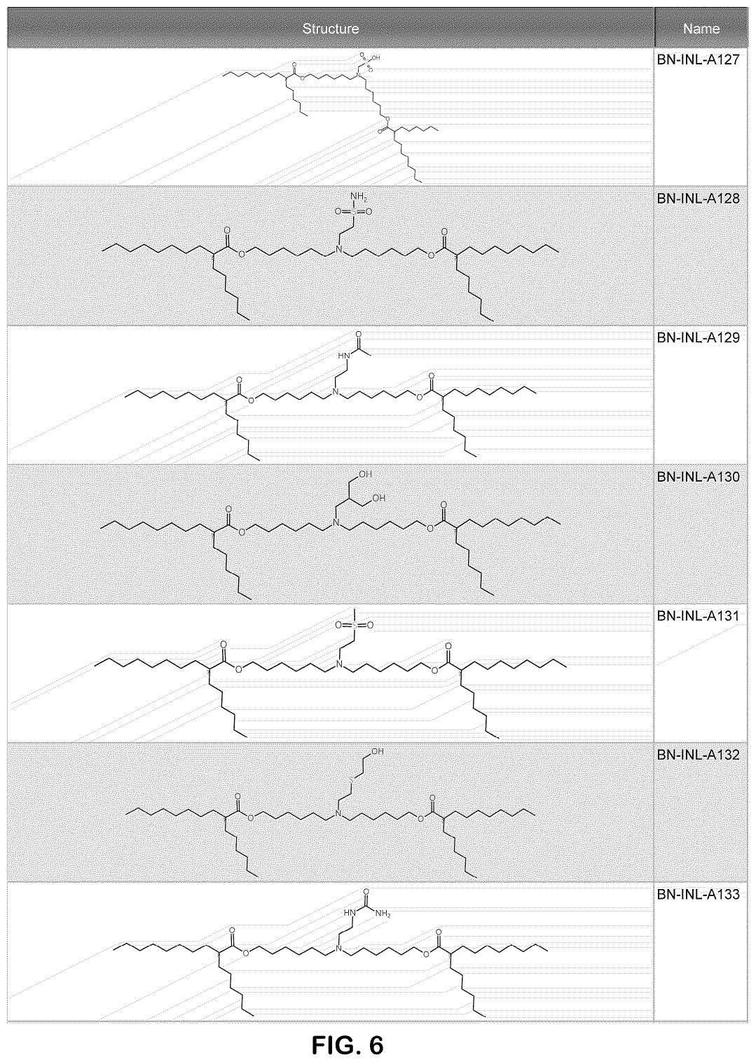

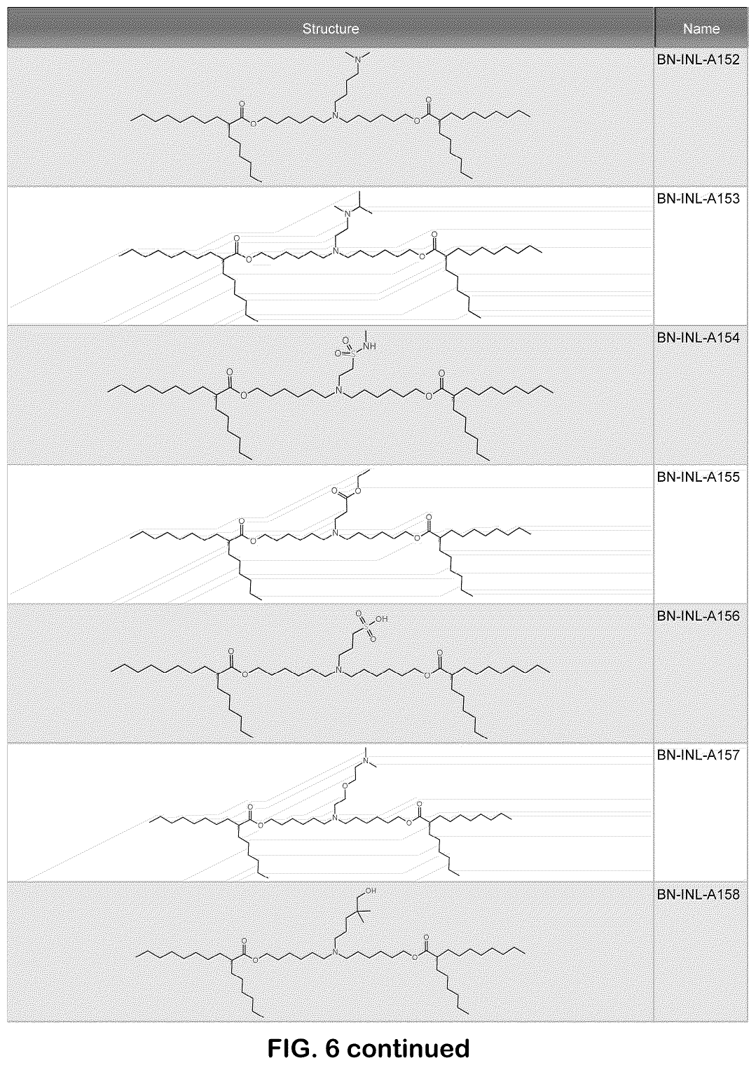

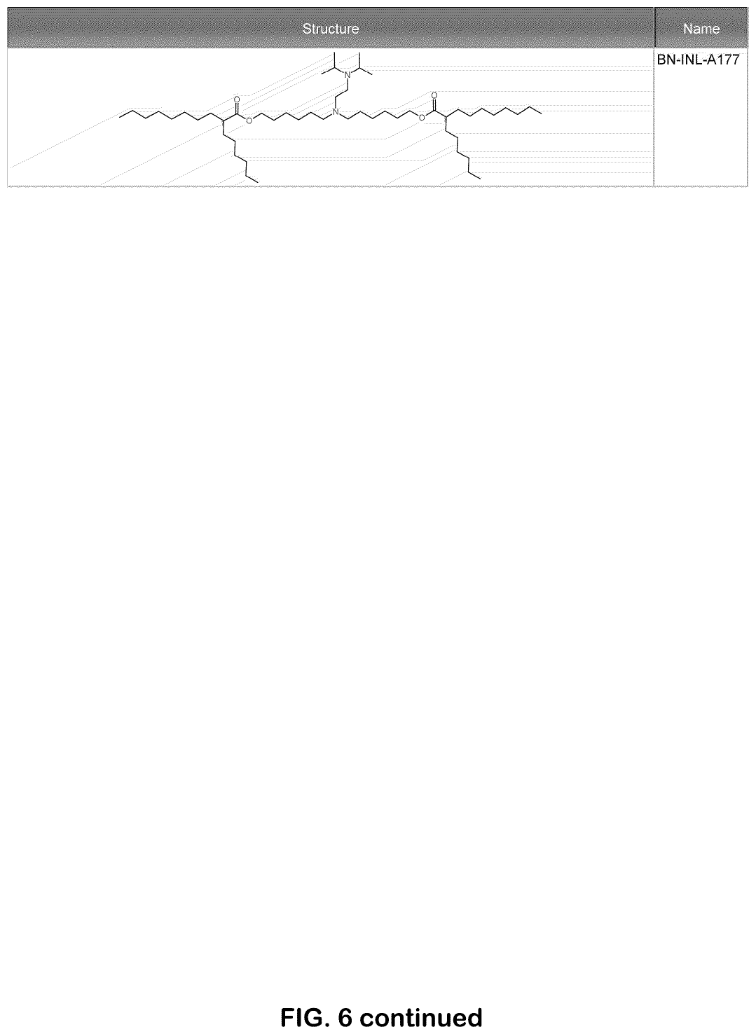

In some embodiments, the compound of the invention represented by Formula 6 is or comprises any of the compounds presented in FIG. 6.

In some embodiments, the compound of the invention comprises any of the compounds of Formulae 1 to 6, and/or any of the compounds of FIGS. 1 to 6, including any salt, any tautomer, and/or any stereoisomer (e.g., an enantiomer, and/or a diastereomer) thereof.

### Carriers

In another aspect, there is provided a carrier for an active agent(s), wherein the carrier is in a form of a core-shell nanoparticle. In some embodiments, the carrier encapsulates the active agent within the core. In some embodiments, the active agent is a small molecule (e.g. an organic molecule with MW below 1000 Da, or below 500 Da), a metal salt (e.g. an inorganic metal salt), and/or a biologic molecule, such as polypeptide (e.g. a protein or a peptide), a polynucleotide, etc., including any combination thereof. In some embodiments, the active agent is selected from a therapeutic agent, a prophylactic agent and a diagnostic agent including any combination thereof. In some embodiments, the one or more active agents are selected from the group consisting of: a protein, a peptide, a nucleic acid (also encompassing a polynucleic acid), a small molecule, a lipid, a glycolipid, and an antibody.

In some embodiments, the term “polynucleic acid” and the term “polynucleotide” are used herein interchangeably. In some embodiments, the polynucleotide comprises 60 to 15000 nucleobases, 1500 to 10000, 1000 to 4700, 200 to 5000 nucleobases, 300 to 5000 nucleobases, 400 to 5000 nucleobases, 400 to 2500 nucleobases, 200 to 3000 nucleobases, 400 to 2000 nucleobases, 400 to 1000 nucleobases, including any range between.

In some embodiments, the polynucleotide comprises at least 20 nucleobases, at least 250 nucleobases, at least 300 nucleobases, at least 350 nucleobases, at least 400 nucleobases, at least 450 nucleobases, at least 475 nucleobases, or at least 500 nucleobases. Each possibility represents a separate embodiment of the invention.

In some embodiments, the polynucleotide comprises 500 nucleobases at most, 750 nucleobases at most, 1,000 nucleobases at most, 1,250 nucleobases at most, 1,750 nucleobases at most, 2,500 nucleobases at most, 3000 nucleobases at most, 4000 nucleobases at most, or 5000 nucleobases at most. Each possibility represents a separate embodiment of the invention.

In some embodiments, the polynucleotide comprises a plurality of polynucleotide types. In some embodiments, the nanoparticle comprises a plurality of polynucleotide types. In some embodiments, the composition comprises a plurality of nanoparticle types, each type of nanoparticle comprises a specific polynucleotide.

In some embodiments, a specific polynucleotide comprises a plurality of polynucleotide molecules harboring the same or an identical nucleic acid sequence. In some embodiments, a specific polynucleotide comprises a plurality of polynucleotide molecules harboring essentially the same nucleic acid sequence.

As used herein, the term “plurality” encompasses any integer equal to or greater than 2. In some embodiments, a plurality comprises at least 2, at least 3, at least 4, at least 5, at least 6, at least 7, at least 8, at least 9, or at least 10, or any value and range therebetween. Each possibility represents a separate embodiment of the invention.

As used herein, the term “polynucleotide types” refers to a plurality of polynucleotides each of which comprises a nucleic acid sequence differing from any one of the other polynucleotides of the plurality of polynucleotides by at least 1 nucleobase, at least 3 nucleobase, at least 5 nucleobase, at least 7 nucleobase, or at least 10 nucleobases, or any value and range therebetween. Each possibility represents a separate embodiment of the invention.

In some embodiments, a polynucleotide comprises RNA, DNA, a synthetic analog of RNA, a synthetic analog of DNA, DNA/RNA hybrid, or any combination thereof. In some embodiments, a nanoparticle of the invention comprises a polynucleotide selected from: RNA, DNA, a synthetic analog of RNA, a synthetic analog of DNA, DNA/RNA hybrid, or any combination thereof.

In some embodiments, the polynucleotide comprises or consists of RNA. The polynucleotide comprises or consists of a messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The basic components of an mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features which serve to overcome existing problems of effective polypeptide expression using nucleic-acid based therapeutics.

The mRNA, as provided herein, comprises at least one (one or more) ribonucleic acid (RNA) polynucleotide having an open reading frame encoding at least one polypeptide of interest. In some embodiments, a RNA polynucleotide of an mRNA encodes 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 polypeptides. In some embodiments, a RNA polynucleotide of an mRNA encodes at least 10, 20, 30, 40, 50, 60, 70, 80, 90 or 100 polypeptides. In some embodiments, a RNA polynucleotide of an mRNA encodes at least 100 or at least 200 polypeptides.

In some embodiments, the nucleic acids are therapeutic mRNAs. As used herein, the term “therapeutic mRNA” refers to an mRNA that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders.

Thus, the carrier of the invention can be used as therapeutic or prophylactic agent. They are provided for use in medicine. For example, the polynucleotide encapsulated within the carrier described herein (e.g. LNP) can be administered to a subject, wherein the polynucleotide is translated in vivo to produce a therapeutic peptide.

In some embodiments, the polynucleotide comprises an inhibitory nucleic acid.

In some embodiments, the polynucleotide comprises an antisense oligonucleotide.

As used herein, an “antisense oligonucleotide” refers to a nucleic acid sequence that is reversed and complementary to a DNA or RNA sequence. It is assumed that, antisense oligonucleotides sterically block a specific DNA or RNA sequence, thereby prevent or at least partially inhibit transcription and/or translation of the specific DNA or RNA sequence, respectively. Exemplary antisense oligonucleotides include a DNA and/or RNA sequence, or comprises a chemically modified backbone/and or base modification within the sequence.

Exemplary chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid (PNA) backbone, a 2-methoxyethyl phosphorothioate backbone, a constrained ethyl backbone, an alternating locked nucleic acid backbone, a phosphorothioate backbone, N3′—P5′ phosphoroamidates, 2′-deoxy-2′-fluoro-3-d-arabino nucleic acid, cyclohexene nucleic acid backbone, tricyclo-DNA (tcDNA) nucleic acid backbone, ligand-conjugated antisense, and a combination thereof.

As referred to herein, a “reversed and complementary nucleic acid sequence” is a nucleic acid sequence capable of hybridizing with another nucleic acid sequence comprised of complementary nucleotide bases. By “hybridize” is meant to form a double-stranded molecule between complementary nucleotide bases (e.g., adenine (A) forms a base pair with thymine (T) (or uracil (U) in the case of RNA), and guanine (G) forms a base pair with cytosine (C)) under suitable conditions of stringency. (See, e.g., Wahl, G. M. and S. L. Berger (1987) Methods Enzymol. 152:399; Kimmel, A. R. (1987) Methods Enzymol. 152:507). For the purposes of the present methods, the inhibitory nucleic acid need not be complementary to the entire sequence, only enough of it to provide specific inhibition; for example, in some embodiments the sequence is 100% complementary to at least nucleotides (nts) 2-7 or 2-8 at the 5′ end of the microRNA itself (e.g., the ‘seed sequence’), e.g., nts 2-7 or 20.

In some embodiments of the inhibitory nucleic acid has one or more chemical modifications to the backbone or side chains. In some embodiments, the inhibitory nucleic acid has at least one chemically modified nucleotide (e.g. LNA, and/or a phosphorothioate).

Non-limiting examples of inhibitory nucleic acids useful according to the herein disclosed invention include, but are not limited to: ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), ribozymes (catalytic RNA molecules capable to cut other specific sequences of RNA molecules) and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro RNA (miRNA); a small, temporal RNA (stRNA); or a short, hairpin RNA (shRNA); small RNA-induced gene activation (RNAa); small activating RNAs (saRNAs), or combinations thereof.

In some embodiments, the inhibitory nucleic acid is an RNA interfering molecule (RNAi). In some embodiments, the RNAi is or comprises double stranded RNA (dsRNA).

As used herein “an interfering RNA” refers to any double stranded or single stranded RNA sequence, capable-either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

In some embodiments, the polynucleotide is chemically modified. In some embodiments, the chemical modification is a modification of a backbone of the polynucleotide. In some embodiments, the chemical modification is a modification of a sugar of the polynucleotide. In some embodiments, the chemical modification is a modification of a nucleobase of the polynucleotide. In some embodiments, the chemical modification increases stability of the polynucleotide in a cell. In some embodiments, the chemical modification increases stability of the polynucleotide in vivo. In some embodiments, the chemical modification increases the stability of the polynucleotide in vitro, such as, in the open air, field, on a surface exposed to air, etc. In some embodiments, the chemical modification increases the polynucleotide's ability to induce silencing of a target gene or sequence, including, but not limited to an RNA molecule derived from a pathogen or an RNA derived from a plant cell, as described herein.

In alternative embodiments, the carrier is in the form of a nanoparticle comprising a shell (or a lipid membrane) encapsulating a core. In some embodiments, the nanoparticle is a lipid nanoparticle. In some embodiments, the carrier encapsulates the active agent within the core. In some embodiments, the shell of the nanoparticle comprises a lipid, and at least one compound of the invention. In some embodiments, the shell of the lipid nanoparticle comprises the compound of the invention. In some embodiments, the shell of the lipid nanoparticle further comprises a lipid, a sterol, and/or a PEG-lipid, or any combination thereof. In alternative embodiments, the carrier is in the form of a lipid nanoparticle comprising the compound of the invention, a lipid and the active agent. In some embodiments, the lipid nanoparticle is in a form of a core-shell nanoparticle, wherein the shell of the nanoparticle comprises a lipid, and at least one compound of the invention.

In some embodiments, the compound of the invention is bound (e.g. via electrostatic interactions) to the active agent (e.g. a polynucleotide). In some embodiments, under suitable conditions the lipid and at least one compound of the invention spontaneously undergo self-assembly in an aqueous solution, so as to form the nanoparticle disclosed herein.

In some embodiments, the term “lipid nanoparticle” refers to a nanoparticle (e.g. substantially spherical particle), wherein the shell of the nanoparticle comprises one or more compounds of the invention and optionally one or more lipids (e.g., a helper lipid, such as a cationic lipid, non-cationic lipid; and optionally a sterol, and/or a PEG-modified lipid). Preferably, the lipid nanoparticles are formulated to deliver one or more agents to one or more target cells.

In some embodiments, the nanoparticle has a spherical geometry or shape. In some embodiments, the nanoparticle has an inflated or a deflated shape. In some embodiments, a plurality of core-shell particles is devoid of any characteristic geometry or shape. In some embodiments, the nanoparticle has a spherical shape, a quasi-spherical shape, a quasi-elliptical sphere, a deflated shape, a concave shape, an irregular shape, or any combination thereof.

In some embodiments, the nanoparticles are substantially spherically shaped, wherein substantially is as described herein. In some embodiments, the nanoparticles are substantially elliptically shaped, wherein substantially is as described herein. One skilled in the art will appreciate that the exact shape of each of the nanoparticles may differ from one particle to another. Moreover, the exact shape of the nanoparticle may be derived from any of the geometric forms listed above, so that the shape of the particle does not perfectly fit a specific geometrical form. One skilled in the art will appreciate that the exact shape of the nanoparticle may have substantial deviations (such as at least 5%, at least 10%, at least 20% deviation) from a specific geometrical shape (e.g., a sphere or an ellipse).

In some embodiments, the lipid is or comprises a phospholipid. In some embodiments, the lipid is or comprises a chemically modified lipid (e.g., a chemically modified phospholipid). In some embodiments, the lipid is or comprises a liposome forming lipid.

In some embodiments, the chemically modified lipid is or comprises a PEG-lipid. In some embodiments, the PEG-lipid comprises a single PEG moiety covalently bound to the head group of the lipid. In some embodiments, the PEG moiety comprises an alkylated PEG such as methoxy poly(ethylene glycol) (mPEG). The PEG moiety can have a molecular weight of the head group from about 750 Da to about 20,000 Da, at times, from about 750 Da to about 12,000 Da and typically between about 1,000 Da to about 5,000 Da, including any range between.

In some embodiments, the shell further comprises a non-liposome forming lipid. When referring to a non-liposome forming lipid it is to be understood as referring to a lipid that does not spontaneously form into a vesicle when brought into an aqueous medium.

There are various types of lipids that do not spontaneously vesiculate and yet are used or can be incorporated into vesicles. In some embodiments, the non-liposome forming lipid is or comprises a sterol.

Non-limiting examples of sterols include but are not limited to: 0-sitosterol, 0-sitostanol, stigmasterol, stigmastanol, campesterol, campestanol, ergosterol, avenasterol, brassicasterol, fucosterol, cholesterol (CHOL), cholesteryl hemisuccinate, and cholesteryl sulfate including any salt or any combination thereof.

In some embodiments, the aqueous core comprises the active agent dissolved or dispersed therewithin. In some embodiments, the nanoparticle comprises a liposomal membrane (e.g., a lipid bilayer).

In some embodiments, a molar concentration of one or more compounds of the invention within the nanoparticle is between 10 and 80 mol %, between 10 and 20 mol %, between 20 and 60 mol %, between 10 and 60 mol %, between 20 and 40 mol %, between 40 and 60 mol %, between 60 and 80 mol %, including any range between. As used herein, the term “concentration” or “molar concentration” refers to a molar ratio relative to the total lipid content of the nanoparticle. In some embodiments, the total lipid content refers to the combined content of the compound of the invention and of the lipid, wherein the lipid encompasses a liposome forming lipid, a modified lipid, and a non-liposome forming lipid. In some embodiments, the total lipid content is substantially located within the shell of the carrier.

In some embodiments, a molar concentration of the lipid within the nanoparticle is between 5 and 40 mol %, between 5 and 10 mol %, between 10 and 40 mol %, between 10 and 30 mol %, between 5 and 20 mol %, between 20 and 40 mol %, including any range between.

In some embodiments, a molar concentration of the sterol within the nanoparticle is between 20 and 60 mol %, between 20 and 30 mol %, between 20 and 50 mol %, between 30 and 60 mol %, between 20 and 30 mol %, between 30 and 50 mol %, between 50 and 60 mol %, including any range between.

In some embodiments, a molar concentration of the modified lipid (e.g., PEG-lipid) within the nanoparticle is between 0.5 and 10 mol %, between 0.1 and 10 mol %, between 0.1 and 0.5 mol %, between 0.5 and 1 mol %, between 1 and 5 mol %, between 5 and 10 mol %, between 5 and 7 mol %, between 7 and 10 mol %, including any range between.

In some embodiments, the carrier is a lipid-based particle. In some embodiments, the carrier is a lipid nanoparticle (LNP). In some embodiments, the carrier is a liposome.

In some embodiments, the carrier is LNP, wherein: a molar concentration of the modified lipid (e.g. PEG-lipid) within the LNP is between 1 and 5 mol %, a molar concentration of the sterol (e.g. cholesterol) within the nanoparticle is between 30 and 40 mol %, a molar concentration of the lipid within the nanoparticle is between 5 and 20 mol %, and a molar concentration of the compound of the invention within the nanoparticle is between 40 and 60 mol %. In some embodiments, the lipid content of the LNP consists essentially of: PEG-lipid at a molar concentration between 1 and 5 mol %, cholesterol at a molar concentration between 30 and 40 mol %, the lipid (e.g. a phospholipid) at a molar concentration between 5 and 20 mol %, and the compound of the invention at a molar concentration between 40 and 60 mol %.

In one embodiment, the carrier is characterized by an average particle size of less than 500 nm to facilitate its entrance through the extracellular matrix to a cell. In one embodiment, the carrier is characterized by an average particle size of less than 300 nm in diameter to facilitate its entrance through the extracellular matrix to a cell.

In one embodiment, the carrier is characterized by an average particle size of less than 300 nm, less than 250 nm, less than 200 nm, less than 150 nm, less than 100 nm, less than 50 nm, less than 20 nm, including any range between.

In another embodiment, the carrier is characterized by an average particle size of between 30 and 300 nm, between 30 and 50 nm, between 50 and 300 nm, between 50 and 250 nm, between 30 and 200 nm, between 50 and 200 nm, between 100 and 300 nm, between 50 and 100 nm, between 100 and 300 nm, between 10 and 1000 nm, including any range between. In another embodiment, the carrier is characterized by an average particle size of between 50 and 300 nm, and by polydispersity index (PDI) below 0.3, below 0.2, or between 0.01 and about 0.3, or between 0.01 and about 0.2.

In some embodiments, the lipid content of the LNP consists essentially of: PEG-lipid at a molar concentration between 1 and 5 mol %, cholesterol at a molar concentration between 30 and 40 mol %, the lipid (e.g. a phospholipid) at a molar concentration between 5 and 20 mol %, and the compound of the invention at a molar concentration between 40 and 60 mol %; and wherein the LNP is characterized by an average particle size of between 50 and 300 nm, and by polydispersity index (PDI) below 0.3, below 0.2, or between 0.01 and about 0.3, or between 0.01 and about 0.2; and further characterized by a negative zeta potential as disclosed below.

In some embodiments, the LNP is a lung-specific LNP, wherein the lipid content of the LNP consists essentially of: PEG-lipid at a molar concentration between 1 and 5 mol %, cholesterol at a molar concentration between 30 and 40 mol %, the lipid (e.g. a phospholipid) at a molar concentration between 5 and 20 mol %, and the compound of any one of Formulae 3-3B (e.g. A159, see FIG. 3) at a molar concentration between 40 and 60 mol %; wherein the lung-specific LNP is characterized by a positive zeta potential (e.g. between +2 and +15 mV); and wherein the lung-specific LNP is characterized by an average particle size of between 80 and 150 nm, and by polydispersity index (PDI) below 0.2.

In another embodiment, the carrier is characterized by a negative zeta potential (e.g., measure at a pH between about 6.5 and 7.5). In some embodiments, the carrier is characterized by a negative zeta potential ranging between −0.1 and −30 mV, between −1 and −20 mV, between −2 and −15 mV, including any range between. In some embodiments, the carrier is characterized by a positive zeta potential ranging between +0.1 and +20 mV, between +2 and +20 mV, between +1 and +20 mV, between +1 and +15 mV, including any range between. The term zeta potential refers to an average value obtained for a specific composition.

In some embodiments, the carrier is stable for a time period ranging between 1 day and 1 year, or more, including any range between. In some embodiments, the term “stable” refers to physical and chemical stability of the carrier (such as being substantially devoid of phase separation, agglomeration, disintegration, and/or substantially retaining the initial loading of the active agent) under appropriate storage conditions. In some embodiments, the term “stable” refers to physical and chemical stability of the carrier within an aqueous solution (e.g., dispersion stability).

In some embodiments, the morphology of the carrier may be spherical or substantially spherical, non-spherical (e.g., elliptical, tubular, etc.), irregular etc.

As used herein, the phrase “lipid nanoparticle” refers to a transfer vehicle, wherein the shell of the carrier comprises one or more lipids (e.g., liposome forming lipids, such as cationic lipids, non-cationic lipids, and PEG-modified lipids) and/or one or more compounds of the invention. Furthermore, the lipid nanoparticles further comprise a non-liposome forming lipid, such as a sterol. Preferably, the lipid nanoparticles are formulated to deliver one or more agents to one or more target cells.

In some embodiments, the carrier comprises a non-cationic lipid, and the compound of the invention. In some embodiments, the carrier comprises a non-cationic lipid, the compound of the invention and a sterol. As used herein, the term “non-cationic lipid” refers to any neutral, or zwitterionic lipid. Non-cationic lipids include, but are not limited to, dioleoylphosphatidylethanolamine (DOPE), distearoylphosphatidylcholine (DSPC), hydrogenated soy phosphatidylcholine (HSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), or a mixture thereof.

In some embodiments, the carrier (e.g., a lipid nanoparticle) is prepared by combining an aqueous phase optionally comprising an active agent, and an organic phase comprising one or more lipid components and the compound of the invention. The selection of specific lipids (such as cationic lipids, non-cationic lipids, sterol(s) and/or PEG-modified lipids) which comprise the lipid nanoparticle, as well as the relative molar ratio of such lipids to each other and/or a molar ratio between the lipid(s) and the compound of the invention, is based upon the characteristics of the selected lipid(s), and the characteristics of the agents to be delivered. Additional considerations include, for example, the saturation of the alkyl chain, as well as the size, Tm, charge, pH, pKa, fusogenicity and toxicity of the selected lipid(s).

In another aspect, there is provided a pharmaceutical composition comprising the lipid nanoparticles of the invention and a pharmaceutically acceptable carrier. In some embodiments, the pharmaceutically acceptable carrier is also referred to an excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition is formulated for systemic administration. In some embodiments, the pharmaceutical composition is formulated for administration to a subject. In some embodiments, the subject is a mammal. In some embodiments, the mammal is a human. In some embodiments, the mammal is a laboratory animal. Examples of laboratory animals include, but are not limited to, mice, rats, rabbits, hamsters, dogs, cats, and monkeys. In some embodiments, the mammal is a mouse or rat. In some embodiments, the subject is in need of the composition. In some embodiments, the subject is in need of treatment. In some embodiments, the subject is a volunteer for a diagnostic method. In some embodiments, the subject is in need of diagnosis.

In some embodiments, the pharmaceutical composition is for use in a therapeutic method. In some embodiments, a therapeutic method is a method of treatment. In some embodiments, the pharmaceutical composition is for use in a diagnostic method. In some embodiments, a diagnostic method is a method of diagnosing. In some embodiments, the pharmaceutical composition is for use in a theranostic method. In some embodiments, a theranostic method is a method of determining a suitable therapeutic for the subject. In some embodiments, the method comprises administering the composition of the invention to a subject.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intratumoral, intramuscular, or intraperitoneal.

The dosage administered will be dependent upon the age, health, and weight of the recipient, kind of concurrent treatment, if any, frequency of treatment, and the nature of the effect desired.

In some embodiments, the composition administered is a composition described in international patent publication WO2016024281 and wherein the composition comprises a lipid of the invention or a nanoparticle of the invention. In some embodiments, the composition administered is a composition described in international patent publication WO2019008590 and wherein the composition comprises a lipid of the invention or a nanoparticle of the invention. In some embodiments, the compositions of the invention are for use in theranostic/diagnostic methods described in WO2016024281, herein incorporated by reference in its entirety. In some embodiments, the compositions of the invention are for use in the theranostic/diagnostic methods described in WO2019008590, herein incorporated by reference in its entirety. In some embodiments, the compositions of the invention are for use in predicting the response of a subject afflicted with a disease to at least one therapeutic agent. In some embodiments, the compositions of the invention are for use in predicting the response of a subject afflicted with a disease to a plurality of therapeutic agents.

By another aspect, there is provided a method of treating a subject in need thereof, the method comprising administering to the subject a therapeutic composition of the invention.

By another aspect, there is provided a method of diagnosing a subject in need thereof, the method comprising administering to the subject a composition of the invention.

In some embodiments, the subject suffers from a disease. In some embodiments, the disease is treatable by the active agent. In some embodiments, the subject is at risk of a disease. In some embodiments, the subject is in need of determining if he/she has a disease. In some embodiments, the subject is in need of determining efficacy of an active agent. In some embodiments, the subject is in need of determining treatment. In some embodiments, determining treatment is determining with which active agent to treat. In some embodiments, determining treatment is determining the dose of active agent with which to treat. In some embodiments, determining treatment is determining the type of nanoparticle with which to treat.

In some embodiments, the method is a diagnostic method and the nanoparticle comprises an active agent and a nucleic acid molecule. In some embodiments, the method is a theranostic method and the nanoparticle comprises an active agent and a nucleic acid molecule. In some embodiments, the nucleic acid molecule uniquely identifies the active agent. In some embodiments, a nanoparticle comprises a particular active agent and a nucleic acid molecule with a particular sequence and the sequence and agent are known. In this way a skilled artisan can identify the active agent by the sequence as a different sequence is used for each different agent. In some embodiments, different agents are different doses of the same agent. In some embodiments, the nucleic acid molecule is a barcode.

In some embodiments, the sequence of the nucleic acid molecule is exclusive of sequences, patterns, signatures or any other nucleic acid sequences associated with a material/substance/particle that is naturally occurring in the environment or particularly naturally occurring in said cell being targeted by the method and composition of the invention. In additional embodiments, the sequence of the nucleic acid molecule is devoid of nucleotide sequences of more than 10 bases which can associate with a naturally occurring nucleotide sequence, and particularly of an exon. In another embodiment, said nucleic acid molecule comprises a sequence which is not substantially identical or complementary to said cell's genomic material (such as to prevent hybridization of the nucleic acid molecule with the cell's genomic material, particularly of said cell's exon and/or prevent false positive amplification results).

In some embodiments, a barcode is short. In some embodiments, short is less than or equal to 150, 100, 90, 80, 60, 50, 45, 40, 35, 30, 25, 20, 15, between 50 and 100, between 80 and 100, between 50 and 200, or 10 bases. Each possibility represents a separate embodiment of the invention. In some embodiments, the barcode is not identical to or complementary to a sequence found in nature. In some embodiments, the barcode does not hybridize to a sequence found in nature. In some embodiments, found in nature is found in the subject. In some embodiments, found in nature is found in a target cell. Barcode sequences and molecules are well known in the art and are commercially available for numerous retailers.

A unique barcode (e.g., a nucleic acid having a unique sequence) is suitable for identifying the corresponding at least one therapeutic agent within the carrier, or the composition of the carrier itself, after implementing the methods of the invention. Methods for the detection of the presence and identification of a nucleic acid sequence are known to a skilled artisan and include PCR and real-time PCR, droplet digital PCR, sequencing and array (e.g., microarray) systems capable of enhancing the presence of multiple barcodes (e.g., commercially available by Ilumina Inc.), alternatively, the presence and identification of a nucleic acid sequence can be performed by detecting the translated protein or peptide within the cell.

In some embodiments, the composition comprises a plurality of types of nanoparticles. In some embodiments, a plurality is at least 2. In some embodiments, a plurality is at least 2, 3, 4, 5, 6, 7, 9, 10, 20, 25, 30, 40, 50, 75, 100, 125, 150, 175, 200, 225, 250, 275, 300, 350, 400, 450 or 500. Each possibility represents a separate embodiment of the invention. In some embodiments, each type of nanoparticle is different from the other. In some embodiments, the plurality comprises at least two nanoparticle types that differ from each other. In some embodiments, different is different in composition. In some embodiments, composition is lipid composition. In some embodiments, different is different in active agent. In some embodiments, different is different in dose of active agent. In some embodiments, different is different in nucleic acid molecule. In some embodiments, different in nucleic acid molecule is different in sequence. In some embodiments, the nucleic acid molecule uniquely identifies the type of nanoparticle.

In some embodiments, the method is a method of determining biodistribution. In some embodiments, biodistribution is biodistribution of the active agent. In some embodiments, biodistribution is biodistribution of the nanoparticle. In some embodiments, a plurality of types of nanoparticles are administered. In some embodiments, the barcodes of each type of nanoparticle uniquely identify the lipid composition of the nanoparticle. That is, various nanoparticles are generated with different lipid compositions and a specific barcode is loaded into each nanoparticle type such that a known barcode identifies each nanoparticle lipid composition. In some embodiments, the barcode sequence is a predetermined sequence. In some embodiments, the barcode sequence is a pre-known sequence. In some embodiments, biodistribution is determined by determining biodistribution of the barcode. In some embodiments, biodistribution is determined by the presence of the barcode. In some embodiments, the presence of a barcode/nucleic acid molecule in a given location in the subject is indicative of a nanoparticle having reached that given location. In some embodiments, a nanoparticle is the nanoparticle identified by the barcode/nucleic acid molecule. In some embodiments, a nanoparticle is the nanoparticle that contained the barcode/nucleic acid molecule. In some embodiments, a location is a tissue. In some embodiments, a location is a cell type. In some embodiments, a location is a disease site. In some embodiments, a location is a tumor. In some embodiments, the biodistribution is determined by the presence of the barcode/nucleic acid molecule.

In some embodiments, the method further comprises receiving a sample from the subject after the administering. In some embodiments, the method further comprises extracting a sample from the subject after the administering. In some embodiments, the sample is from the location. In some embodiments, the location is a plurality of locations. In some embodiments, the sample is from a location to be investigated for distribution. In some embodiments, the sample is tissue. In some embodiments, the sample is fluid. In some embodiments, the sample is a tumor. In some embodiments, the sample comprises disease cells.

In some embodiments, the method further comprises extracting nucleic acids from the sample. In some embodiments, the method further comprises purifying the nucleic acid molecule. Methods of nucleic acid extraction, isolation and purification are well known in the art and any such method may be employed. In some embodiments, the nucleic acid molecules are analyzed. In some embodiments, the nucleic acid molecules are quantified. In some embodiments, analyzed is analyzed for sequence. In some embodiments, the nucleic acid molecules are sequenced. In some embodiments, sequencing is deep sequencing. In some embodiments, sequencing is next generation sequencing.

### Definitions

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”.

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)₂R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R″ end group or a —R′NC(N) NR″— linking group, as these phrases are defined hereinabove, where R′, R″ and R″ are as defined herein. As used herein, the term “azide” refers to a —N3 group. The term “sulfonamide” refers to a —S(O)₂NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C₃₋₈ heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C₁₋₆-alkyl, C₁₋₆-haloalkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C₁₋₆-alkyl, C₁₋₆-alkyl, hydroxy-C₁₋₆-alkyl and amino-C₁₋₆-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

### General

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description or claims, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

The inventors presume that the compounds of the invention can be utilized for the preparation of stable LNPs. A composition of an exemplary LNP may be as follows:
phospholipid (e.g., DSPC and/or DOPE) about 5-50 mol %; sterol (e.g., cholesterol) about 10-50 mol %; compound of the invention about 10-60 mol %; and optionally a PEG lipid about 1-20 mol %. The LNPs can be characterized by an average particle size ranging between about 10 and about 300 nm.

For the preparation of exemplary LNPs, two phases: an organic and an aqueous phase can be prepared. The organic phase should contain the lipids (including the liposome-, and optionally non-liposome forming lipids and the compound of the invention) and the aqueous phase should contain the active agent (e.g., a polynucleic acid). These phases can be rapidly mixed together at a low pH, e.g. via microfluidics.

The solution can be brought to neutral pH to obtain LNPs. The LNPs can be characterized by measuring the size using dynamic light scattering and zeta potential using electrophoretic mobility. The compositions of the LNPs can be checked via quantitative HPLC. Here it is possible to test the stability of the lipids within the LNP by looking for novel peaks corresponding to breakdown products or by assessing a possible leakage of the active agent. It is also possible to test the LNPs stability over time using the HPLC method. The encapsulation efficiency can be calculated by subtracting the amount of non-encapsulated nucleic acid form the total nucleic acid content. The amount of the non-encapsulated nucleic acid can be determined via a fluorescence assay. The total concentration of the active agent can be confirmed via digital PCR.

The biodistribution of the exemplary LNPs can be determined in-vivo, by injecting the LNPs into mice. The delivered nucleic acids from various organs of these mice can be extracted and the copy number can be calculated by digital PCR.

### Example 2

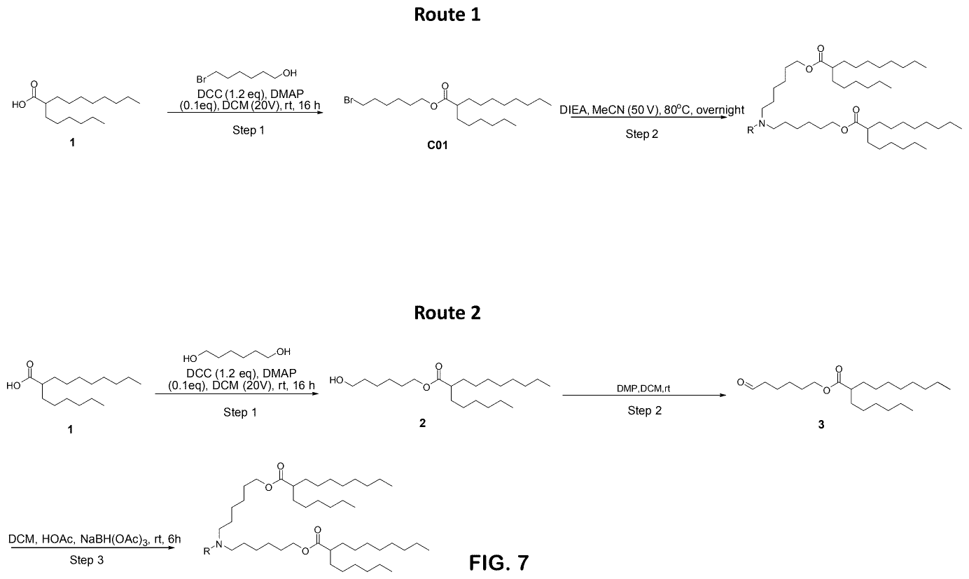

The inventors successfully synthesized a list of exemplary ionizable lipids of the invention (see FIG. 1-6) representing the compounds of Formulae 1-6. The entire compounds have been synthesized according to exemplary procedures (Route 1 or Route 2) depicted in FIG. 7.

From the initial list of exemplary ionizable lipids, 6 lipids each representing the compounds of Formulae 1-6, successfully demonstrated LNP formation of an aqueous solution including an oligonucleotide (a reporter luciferase mRNA as well as barcode DNA) encapsulated therewithin. The LNPs were manufactured as disclosed in Example 1.

Table 1 below presents the chemical composition of the tested exemplary formulations along with the internal ID of each corresponding ionizable lipid.

TABLE 1

| Formulation ID | Ion- | Helper | PEG₂₀₀₀ | Molar Composition | | |
|---|---|---|---|---|---|---|
| and corresponding | Lipid | lipid (mol | lipd (mol | (%) | | |
| Formula ( ) | name | %) | %) | Ion-lipid | Cholesterol | pKa |
| 40-1 (1) | A141 | DSPC | DMG (1.5) | 50 | 38.5 | 4.97 |
| | | (10) | | | | |
| 40-2 (2) | A138 | DSPC | DMG (1.5) | 50 | 38.5 | 5.96 |
| | | (10) | | | | |
| 40-3/40-7 (3) | A167/159 | DSPC | DMG (1.5) | 50 | 38.5 | 5.50 |
| | | (10) | | | | |
| 40-4 (4) | A200 | DSPC | DMG (1.5) | 50 | 38.5 | 7.48 |
| | | (10) | | | | |
| 40-6 (6) | A129 | DSPC | DMG (1.5) | 50 | 38.5 | 5.89 |
| | | (10) | | | | |
| 40-8 (5) | A212 | DSPC | DMG (1.5) | 50 | 38.5 | 4.65 |
| | | (10) | | | | |

The pKa of 40-7 was above 10.

The N/P ratio of the tested formulations was 8. All the tested ionizable lipids of Table 1 successfully formed LNPs with an average particle size between about 80 and about 220 nm with a narrow PDI between 0.04 and about 0.2, as determined by Z-sizer. Furthermore, the tested formulations exhibited a negative zeta potential ranging between −4 and −12 mV, except formulation 40-7, having a positive zeta potential of about +10 mV.

Additionally, a concentration of the encapsulated oligonucleotide in the tested formulations was between about 50-120 ng/ul.

### Example 3

The inventors aimed to determine in-vivo performance of some of the formulations listed in Table 1. For this purpose, inventors encapsulated Luciferase mRNA in different formulations using the ionizable lipids of the invention, as listed in Table 1. The inventors injected mice with each one of the LNP formulations at a concentration corresponding to approximately 0.5 mg encapsulated mRNA/kg. As a control, a free mRNA+barcode was used.

BAlb/c female mice (8 weeks old) were injected with the different formulations (n=2). 6 hours later, Luminescence signal emanating from expression of the mRNA in a panel of tissues was recorded and analyzed using IVIS.

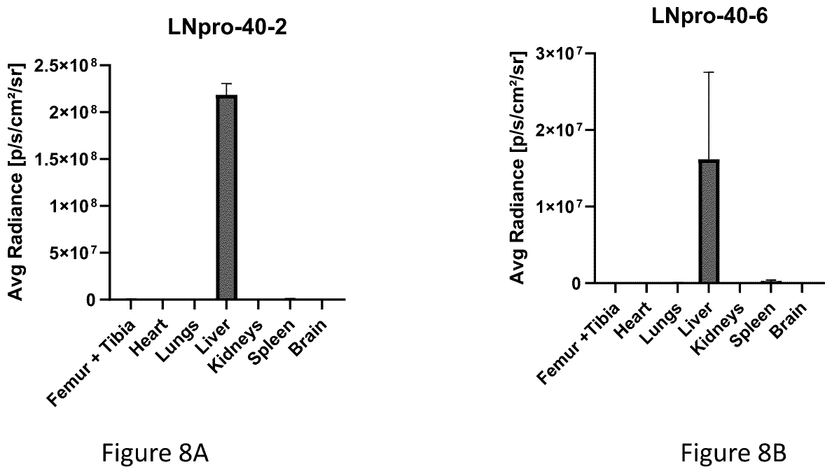

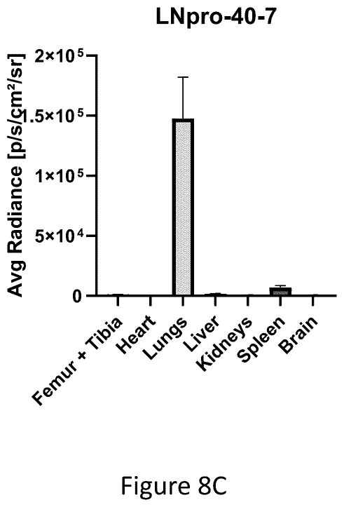

At endpoint, animals were anaesthetized, and live luminescence was recorded using IVIS imaging at the organ scale. The results of organ specific distribution of the tested formulations are presented in FIGS. 8A-8C.

Surprisingly, formulation 40-7 (including the compound A-159 as the ionizable lipid) showed a highly lung-specific in-vivo biodistribution. Additionally, formulations 40-6 and 40-2 showed specific liver distribution. Interestingly, formulation 40-8 showed a broad distribution with high delivery to the lung, spleen and bones as well as moderate delivery to the heart, kidneys and brain.

To this end, the compounds of the invention showed markable LNP (encapsulating an oligonucleotide) formation ability. Furthermore, based on in-vivo data, exemplary LNPs of the invention can be utilized for administration of an oligonucleotide payload to a subject in need thereof. Moreover, exemplary LNPs of the invention can be utilized for a targeted delivery of the oligonucleotide payload to a specific location (e.g. to the lung using compound A-159).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A compound represented by Formula 3: 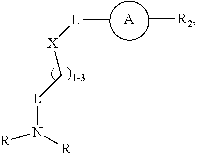
wherein A represents a 5-membered heteroaryl, 5-membered cycloalkyl optionally comprising an unsaturated bond, or a 5-membered heterocyclyl optionally comprising an unsaturated bond, or by Formula 1: 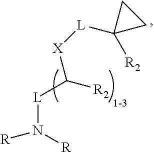
or Formula 2: 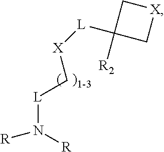
or by Formula 4: 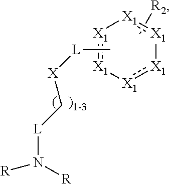
wherein each X₁ independently comprises CH, CH₂, CHR₂, CHR₁, NR₁, NR₂, NH, N, O, or S;
or by Formula 5: 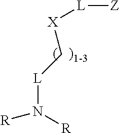
wherein Z represents (i) an optionally substituted bicyclic aliphatic ring optionally comprising between 1 and 4 X₁, or (ii) an optionally substituted bicyclic ring optionally comprising between 1 and 4 X₁, wherein at least one ring is aromatic,
or by Formula 6: 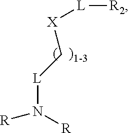
wherein each R independently comprises any of: 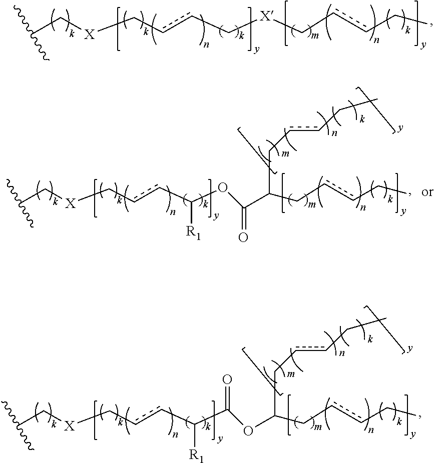
wherein k,
and y, are integers each independently being between 1 and 10; n and m are integers each independently being between 0 and 10; each X independently comprises CH₂, CHR₂, CHR₁, C(R₂)₂, C(R₁)₂, NR₁, NR₂, NH, O, S, or is absent; X′ represents CH₂, CHR₂, CHR₁, O, S, —CONH(R′), —CON(R′)₂, —CO₂R′, —OCOR′, —OC(═O)O—, —OC(═O)NR′, —OC(═S)OR′, —OC(═S)NR′, —SO₂—, —SO—, —SO₂O—, —SO₂NR′; each L independently is absent, or represents a (C1-C5) alkyl, an ester, a carbonyl, an amide, CHR₁, C(R₁)₂, NR₁, NH, O, S, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂—, —CO₂R′, —OCO—, —OCOR′, —OC(═O)O—, —OC(═O)NR′, —OC(═S)OR′, —OC(═S)NR′, —SO₂—, —SO—, —SO₂O—, —SO₂NR′, or any combination thereof as allowed by valency; wherein each R₁ is H or independently comprises (i) a C1-C30 alkyl, (ii) a C1-C30 alkenyl, (ii) a C1-C30 alkynyl, each of (i), (ii) and (iii) optionally comprising one or more heteroatoms; and wherein each R₂ is independently absent, or represents one or more substituents selected from hydrogen, halogen, —NO₂, —CN, —OH, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C1-C30 alkyl, optionally substituted C1-C30 alkenyl, optionally substituted C1-C30 alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, or a combination thereof.

2. The compound of claim 1, wherein said compound is represented by Formula 3A: 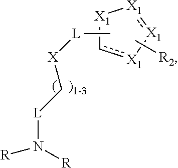
wherein each X₁ independently comprises CH, CH₂, CHR₂, CHR₁, NR₁, NR₂, NH, O, N, or S;
or by Formula 3B: 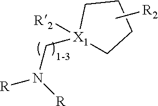
wherein X1 is C or N, as allowed by valency; and
wherein R′₂ is selected from H, hydroxy and hydroxy(C₁-C₆ alkyl); optionally wherein said compound is or comprises any one of compounds of FIG. 3.

3. (canceled)

4. (canceled)

5. (canceled)

6. (canceled)

7. (canceled)

8. (canceled)

9. (canceled)

10. (canceled)

11. The compound of claim 1, wherein Z comprises 7-12 membered bicyclic ring optionally substituted with one or more R₂; optionally wherein the 7-12 bicyclic ring optionally comprises between 1 and 4 X₁, including any range between.

12. (canceled)

13. (canceled)

14. (canceled)

15. (canceled)

16. The compound of claim 1,
wherein each R is independently 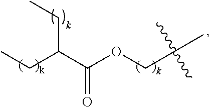
optionally
wherein each k is between 4 and 6, including any range between.

17. (canceled)

18. A nanoparticle comprising a core and a shell:
(i) the shell comprises a lipid, and at least one compound of claim 1;
(ii) the core comprises an active agent;
and wherein an average size of the nanoparticle is in a range between 10 and 1000 nm.

19. The nanoparticle of claim 18, wherein a molar concentration of the compound within the nanoparticle is between 10 and 80 mol % wherein the nanoparticle further comprises a sterol; wherein a molar concentration of the sterol within the nanoparticle is between 20 and 60 mol %.

20. (canceled)

21. (canceled)

22. The nanoparticle of claim 18, wherein a molar concentration of the lipid within the nanoparticle is between 5 and 70 mol % wherein said lipid is a liposome forming lipid; and
wherein said lipid further comprises a PEG-lipid.

23. (canceled)

24. (canceled)

25. The nanoparticle of claim 18, wherein said nanoparticle is a lipid nanoparticle.

26. A pharmaceutical composition comprises a plurality of the nanoparticles of claim 18 and a pharmaceutically acceptable carrier.

27. The pharmaceutical composition of claim 26, comprising an effective amount of the active agent, and formulated for systemic administration, administration to a subject, or both; said pharmaceutical composition optionally comprising a plurality of types of nanoparticles wherein said plurality comprises at least two nanoparticle types that differ in their lipid composition, their active agent, their nucleic acid molecule or a combination thereof; optionally wherein said nucleic acid molecule uniquely identifies each nanoparticle type.

28. (canceled)

29. (canceled)

30. (canceled)

31. (canceled)

32. A method for delivering of an active agent to a tissue of a subject, comprising administering to the subject the pharmaceutical composition of claim 26, wherein said active agent is a polynucleic acid.

33. (canceled)

34. The method of claim 32, wherein said method comprises administering a therapeutically effective amount of said pharmaceutical composition.

35. The method of claim 32, wherein said tissue is selected from brain tissue, lung tissue, spleen tissue, liver tissue, kidney tissue, bone tissue and heart tissue; and wherein the plurality of the nanoparticles in the pharmaceutical composition comprise between 30 and 60 mol % of the compound of Formula 5, and optionally further comprise the PEG-lipid at a molar concentration between 1 and 5 mol %, cholesterol at a molar concentration between 30 and 40 mol %, and the lipid at a molar concentration between 5 and 20 mol %.

36. The method of claim 35, wherein the pharmaceutical composition comprises any one of: 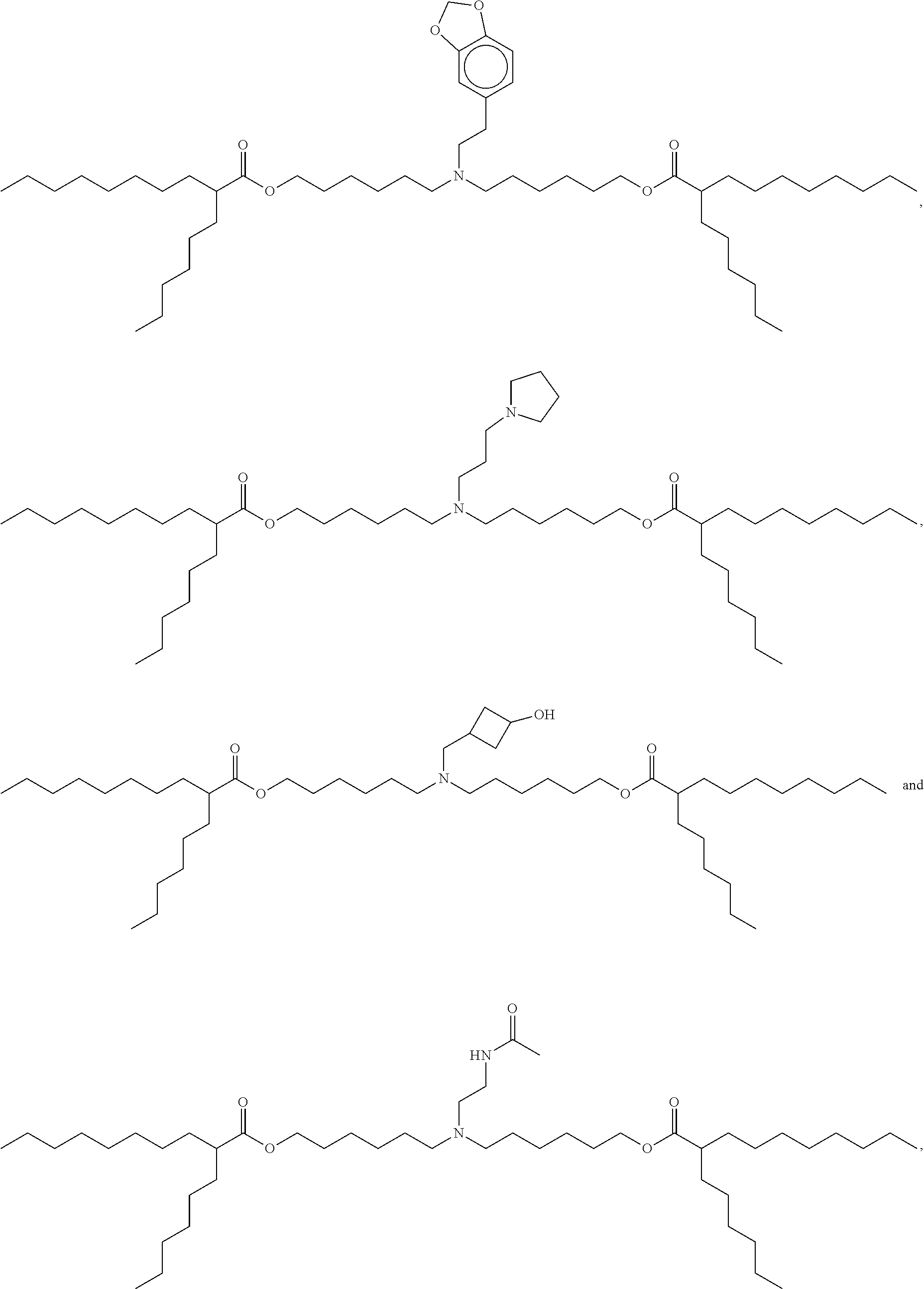
including any salt thereof; and wherein the pharmaceutical composition is characterized by an average particle size between 80 and 250 nm and by a PDI below 0.2; and optionally is characterized by negative average zeta potential.

37. The method of claim 32, wherein said tissue is a lung tissue; wherein the plurality of the nanoparticles in the pharmaceutical composition comprise between 30 and 60 mol % of the compound of any one of Formulae 3-3B, and optionally further comprise the PEG-lipid at a molar concentration between 1 and 5 mol %, cholesterol at a molar concentration between 30 and 40 mol %, and the lipid at a molar concentration between 5 and 20 mol %.

38. (canceled)

39. The method of claim 32, wherein said tissue comprises liver tissue; wherein the plurality of the nanoparticles in the pharmaceutical composition comprises between 30 and 60 mol % of the compound of Formula 2 or of Formula 6, and optionally further comprise the PEG-lipid at a molar concentration between 1 and 5 mol %, cholesterol at a molar concentration between 30 and 40 mol %, and the lipid at a molar concentration between 5 and 20 mol %.

40. (canceled)

41. The compound of claim 1, wherein the compound comprises any one of: 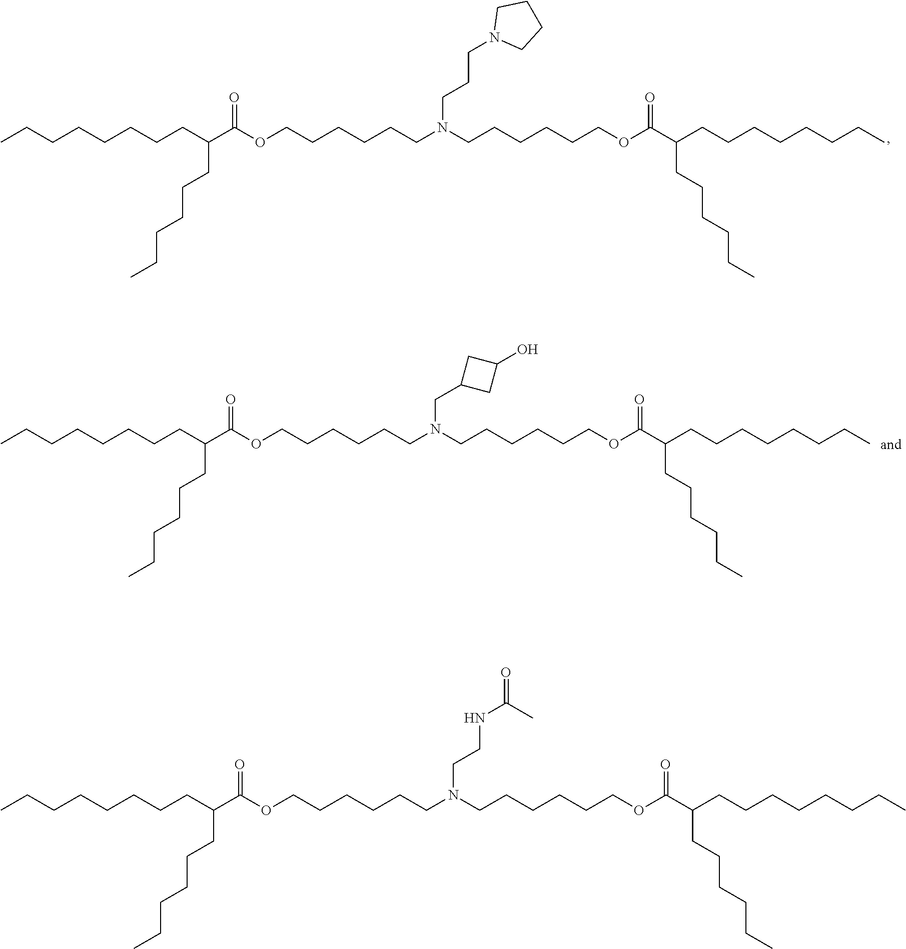
including any salt thereof.

42. (canceled)

43. (canceled)
